(19) United States
(12) Patent Application Publication
(10) Pub. No.: US 2007/0092586 A1
(43) Pub. Date: Apr. 26, 2007

(54) Compositions and methods for the administration psychotropic drugs which modulate body weight

(75) Inventor: Neal R. Cutler, Beverly Hills, CA (US)

Correspondence Address: KNOBBE MARTENS OLSON & BEAR LLP, 2040 MAIN STREET, FOURTEENTH FLOOR, IRVINE, CA, 92614, US

(73) Assignee: Alamo Pharmaceuticals, Beverly Hills, CA (US)

(21) Appl. No.: 11/362,495

(22) Filed: Feb. 24, 2006

Related U.S. Application Data
(63) Continuation-in-part of application No. 11/258,784, filed on Oct. 26, 2005.

(51) Int. Cl. A61K 33/00; A61K 31/551; A61K 31/506; A61K 31/496; A61K 31/137; A61K 31/445

(52) U.S. Cl. 424/722; 514/220; 514/252.15; 514/317; 514/469; 514/649; 514/253.04

(57) ABSTRACT

Embodiments of the invention describe compositions and methods for the administration of fast disintegrating atypical antipsychotics, metabolites of atypical antipsychotics, and antidepressants which reduce weight in patients previously taking conventional formulation of atypical antipsychotics and antidepressants. In a preferred embodiment said fast dissolving atypical antipsychotic is FAZACLO. In another preferred embodiment said fast dissolving metabolite of an atypical antipsychotic is desmethyl clozapine. In another preferred embodiment said fast dissolving antidepressant is paroxetine.

### FIELD OF THE INVENTION

The present invention describes the administration of atypical antipsychotics, the metabolites of atypical antipsychotics, and antidepressant formulations that reduce body weight in patients who are overweight or obese secondary to treatment with certain psychotropic drugs. The invention also contemplates embodiments wherein patients, who are candidates for treatment with atypical antipsychotics, the metabolites of atypical antipsychotics, and antidepressants and have not been previously treated with conventional formulations of these same drugs, are treated with the fast dissolving formulations of the present invention under conditions such that they do experience weight gain which may be associated with the administration of conventional formulations of these same drugs.

### BACKGROUND

Weight gain has been well recognized as having significant physical and psychological consequences. Increased body weight and obesity are associated with chronic diseases such as hypertension, coronary heart disease and diabetes mellitus. Substantial weight gain may also adversely affect self-esteem, social functioning and physical activity. Obesity is rightly seen to be a major public health concern throughout the world. The estimated economic burden of obesity to the United States alone is about $117 billion annually, and obesity is associated with an estimated 300,000 deaths per year. Further, numerous diseases have been correlated to obesity: heart disease, certain types of cancer, sleep apnea, asthma, arthritis, pregnancy complications, depression and type II diabetes mellitus.

Drug-induced weight gain has also been long recognized, particularly as a consequence from the use of psychotropic medication. Although weight gain has been a documented adverse effect of atypical antipsychotics for more than a decade, it has received surprisingly little attention. This is an important observation, since atypical antipsychotics are considered to provide major advantages over many conventional antipsychotic drugs.

Undesired weight gain is a common complaint of patients receiving pharmacological treatment for depression. These include, but are not limited to, first generation tricyclic compounds (ELAVIL, for example) and more contemporary selective serotonin reuptake inhibitors (SSRIs) including, but not limited to PAXIL and REMERON. Specifically, tricyclic antidepressants (ELAVIL, for example) were found to stimulate appetite, carbohydrate craving, and a dose-dependent continuous weight gain of 0.57 to 1.37 kg per month of treatment. Selective serotonin reuptake inhibitors (SSRIs) including, but not limited to, PAXIL and REMERON are also associated with weight gain.

Monoamine oxidase inhibitors may also provoke weight gain by stimulating appetite and potentiating insulin-induced hypoglycemia. In addition, lithium maintenance therapy stimulates weight gains of over 10 kg in 20% of patients. See, for example, J. Clin. Psychopharmacol. 1988 Oct.; 8(5): 323-30.

Nonetheless the weight gain and, in many cases, subsequent chronic morbid obesity associated with the administration of atypical antipsychotics, the metabolites of atypical antipsychotics and antidepressants often cause patients to abandon, or causes their physicians to change, the therapeutic agent effective in controlling their depression and/or psychosis. What is needed, therefore, are formulations of proven antidepressants and atypical antipsychotics that are not associated with the morbidities described above.

### SUMMARY OF INVENTION

The present invention relates to methods for the administration of fast disintegrating clozapine formulations which exert a therapeutic antipsychotic effect without inducing the degree of weight gain typically observed with other psychotropic drugs including, but not limited to, conventional formulations of atypical antipsychotics. Embodiments of the present invention also describe weight loss in patients previously treated with clozapine, including but not limited to formulations such as CLOZARIL (Novartis) and Clozapine Tablets (Mylan), who change over to the clozapine formulations described in various embodiments of the present invention. The formulations of the present invention are broadly classified as “fast disintegrating”. While it is not intended the present invention be limited to any specific formulation, in a preferred embodiment, said fast disintegrating formulation is an ODT (Orally Disintegrating Tablet) marketed under the trade name FAZACLO (Alamo Pharmaceuticals).

The present invention also relates to methods for the administration the metabolites of clozapine as a fast disintegrating formulation. It is not intended that the present invention be limited to any specific metabolite of clozapine. In one embodiment, the present invention contemplates the compounds labeled 1-14, as set out in FIG. 1., will be formulated into a fast disintegrating formulation. In a preferred embodiment, desmethyl clozapine (having the IUPAC name of: 8-Chloro-11-(1-piperazinyl)-5H-dibenzo[b,E][1,4]diazepine) will be formulated into a fast disintegrating formulation.

Embodiments of the present invention also describe weight loss in patients previously treated with clozapine, including but not limited to formulations such as CLOZARIL (Novartis) and Clozapine Tablets (Mylan), who change over to the fast disintegrating formulation of the present invention which have one or more metabolites of clozapine as the active ingredient. While it is not intended the present invention be limited to any specific formulation, in a preferred embodiment, said fast disintegrating clozapine metabolite formulations are an ODT (Orally Disintegrating Tablet) as developed by Cima Laboratories. In a preferred embodiment said ODT is formulated with desmethyl clozapine. It is not intended that the present invention be limited to any specific dosage of clozapine metabolites in a given dosage form. However, in a preferred embodiment, the dosage of desmethyl clozapine, per ODT, is in the range between 12.5 mg and 1,800 mg.

In other embodiments, the present invention also relates to methods for the administration of fast disintegrating formulations of antidepressants which exert a therapeutic effect without inducing the degree of weight gain typically observed with conventional formulations of these same antidepressants. Embodiments of the present invention also describe weight loss in patients previously treated with antidepressants, who change over to the fast disintegrating formulations described in various embodiments of the present invention. While it is not intended the present invention be limited to any specific formulation, in a preferred embodiment, said fast disintegrating formulation is an ODT (Orally Disintegrating Tablet) as developed by Cima Laboratories. While it is not intended that the present invention be limited to any specific antidepressant, in preferred embodiments fast disintegrating formulations of the following drugs are contemplated: citalopram (CELEXA, CIPRAMIL); escitalopram oxalate (CIPRALEX, LEXAPRO); fluvoxamine maleate (LUVOX); paroxetine (PAXIL, SEROXAT, AROPAX); fluoxetine (PROZAC); sertraline (ZOLOFT, LUSTRAL); amitriptyline (ELAVIL, ENDEP); clomipramine (ANAFRANIL); desipramine (NORPRAMIN, PERTOFRANE); doxepin (ADAPIN, SINEQUAN); imipramine (TOFRANIL); nortryp-tyline (PAMELOR); protriptyline (VIVACTIL); trimipramine (SURMONTIL); maprotiline (LUDIOMIL); bupropion (WELLBUTRIN); buspirone (BUSPAR); duloxetine (CYMBALTA); mirtazapine (REMERON, ZISPIN, AVANZA, NORSET, REMERGIL); nefazodone (SERZONE); reboxetine (EDRONAX, VESTRA); trazodone (DESYREL); venlafaxine (EFFEXOR); phenelzine (NARDIL), tranylcypromine (PARNATE), and lithium carbonate. The present invention also contemplates co-formulating two or more of the antidepressants set out above in a fast disintegrating formulation.

It is not intended that the present invention be limited to any specific dosage of antidepressants. In preferred embodiment, however, the present invention contemplates ODTs, to be administered once-a-day, having the dosages of antidepressant as set out in Table 1.

TABLE 1
Preferred Dosages For An Exemplar Group Of Antidepressants

| Antidepressant | Dosage Per ODT | |
|---|---|---|
| Bupropion (Wellbutrin) | 300 to 400 | mg |
| Fluoxetine (Prozac) | 20 to 80 | mg |
| Mirtazapine (Remeron) | 15 to 30 | mg |
| Nefazadone (Serzone) | 200 to 600 | mg |
| Paroxetine (Paxil) | 10 to 40 | mg |
| Sertraline (Zoloft) | 50 to 200 | mg |
| Phenelzine | 15 mg to 100 | mg |
| Tranylcypromine | 10 mg to 60 | mg |
| Amitriptyline | 50 mg to 200 | mg |

In other embodiments, the present invention also relates to methods for the administration of fast disintegrating formulations of clozapine metabolites which exert a therapeutic effect without inducing the degree of weight gain typically observed with conventional formulations of atypical antipsychotics. Embodiments of the present invention describe weight loss in patients previously treated with an atypical antipsychotic, who change over to an ODT (Orally Disintegrating Tablet) comprising desmethyl clozapine. The present invention also contemplates a fast disintegrating formulation comprising clozapine and/or a metabolite of clozapine.

In a retrospective review, Leppig and colleagues (Leppig M. et al. “Clozapine in the Treatment of 121 Out-Patients.” Psycopharmacology 1989; 99: S77±S79) reported ‘weight gain’ occurring in 23% of patients given clozapine for an average of nearly 3 years. Similarly, Gerlach and co-workers (Gerlach J. et al. “Long-term Experience with Clozapine in Denmark: Research and Clinical Practice.” Psycopharmacology 1989; 99: S92±S96) noted a significant (P<0.05) increase in body weight (mean=2.8 kg) in 59 patients taking clozapine for an unstated period. Substantial weight gain associated with clozapine was later reported in six of seven patients treated with the drug (Cohen S. et al. “A Weight Gain Associated with Clozapine.” Am. J. Psychiatry 1990; 147: 503±504). The latter research group reported their findings in full 2 years later, noting that eight of 21 patients taking clozapine gained more than 10% of baseline weight over 16 weeks. These observations were supported by those of Lamberti et al. (Lamberti J S, et al. “Weight Gain Among Schizophrenic Patients Treated With Clozapine.” Am. J. Psychiatry 1992; 149: 689±690) who reported substantial weight gain in small case series, and by Umbricht et al. (Umbricht D S G, et al. “Clozapine and Weight Gain.” J Clin Psychiatry 1994; 55 (Suppl B): 157±160), who reviewed retrospectively 82 patients taking clozapine for up to 90 months (60% gained>10% of baseline weight in 12 months).

More recently, substantial weight gain has been unequivocally associated with the use of clozapine. John and co-workers (John J P et al., Assessment of Changes in both Weight and Frequency of Use of Medications for the Treatment of Gastrointestinal Symptoms among Clozapine-Treated Patients.” Ann Clin Psychiatry 1995; 7; 119±125) measured weight gain of more than 10% of original weight in 27% of patients taking clozapine for 3 months or more. Jalenques et al. (Jalenques I., et al., “Weight Gain as a Predictor of Long Term Clozapine Efficacy.” Clin Drug Invest 1996; 12: 16±25) noted a mean increase in body weight of 12.4 kg over 21 months in patients who responded to clozapine and remained clinically stable. The mechanism by which clozapine promotes obesity and diabetes are not well described. However investigators have suggested these co-morbidities could involve: suppression of insulin release, insulin resistance, or impairment of glucose utilization. Some investigators suggest that clozapine interaction with specific serotonin receptors may also contribute to these metabolic abnormalities. Other investigators have suggested the dramatic increase in food consumption exhibited by many patients on clozapine is attributable to clozapine's interaction with satiety receptors in the stomach (receptors for cholecystokinin, in one example). This interaction causes, in some cases, patients who take clozapine to eat continuously without experiencing a sense of fullness.

While it is not intended the present invention be limited to any specific mechanism, the Applicant believes the fast disintegrating formulations described by embodiments of the present invention present atypical antipsychotics to the gastrointestinal tract (hereinafter referred to as the “GI tract”) under conditions such that normal satiety feedback is not disrupted. Satiety signals originate in the GI tract. The Applicant believes that conventional formulations of atypical antipsychotics, in one example CLOZARIL, and metabolites of atypical antipsychotics, in one example desmethyl clozapine, present atypical antipsychotics and their metabolites to the stomach lining as a bolus of drug which interacts with satiety receptors in the stomach under conditions such that a patient continues to eat without feeling a sense of fullness. The extra caloric intake associated with this “over eating” promotes weight gain in patients taking these conventional formulations of atypical antipsychotics.

In contrast, the fast disintegrating formulations described by embodiments of the instant invention present atypical antipsychotics, in a preferred embodiment FAZACLO, and metabolites of atypical antipsychotics, in one example an ODT comprising desmethyl clozapine, to the stomach in a metered fashion. That is to say the fast disintegrating formulations of atypical antipsychotics and their metabolites, described by embodiments of the present invention, are converted into a slurry when contacted with the saliva in the mouth. This drug and saliva slurry is delivered to the stomach via the reflexive swallowing of the saliva. The Applicant believes this change, as compared to conventional formulations of atypical antipsychotics and their metabolites in the way the fast disintegrating formulations of the present invention are delivered to the stomach prevents (or reduces) interference with satiety receptors in the stomach. A patient taking any of the fast disintegrating formulations described by embodiments of the present invention will experiences a sense of fullness upon completing a meal and, therefore, is less likely to experience weight gain as a function of overeating. It is not intended that the present invention be limited to any specific dose of atypical antipsychotic. In one embodiment, clozapine is administered in a range between 12.5-900.0 mg per day. In a preferred embodiment clozapine is administered in a range between 50-600 mg per day.

In one embodiment, the present invention comprises treating a patient suffering from one or more symptoms of psychosis with a therapeutic formulation of clozapine, or a metabolite of clozapine, that does not promote weight gain in a patient with a substantially normal BMI. It is contemplated that oral administration of clozapine, or a metabolite of clozapine, may be made with a fast disintegrating formulation. In one embodiment this fast disintegrating formulation is an ODT. In a preferred embodiment the ODT comprising clozapine is FAZACLO.

In one embodiment the present invention describes a hard, compressed, fast disintegrating tablet adapted for oral administration. The tablet includes particles made of an active ingredient and a protective material. These particles are provided in an amount of between about 0.01 and about 75% by weight based on the weight of the tablet. The tablet may also include a matrix made from a nondirect compression filler, a wicking agent, and a hydrophobic lubricant. The preferred tablet matrix comprises at least about 60% rapidly water-soluble ingredients based on the total weight of the matrix material. The preferred tablet has a hardness of between about 15 and about 50 Newtons, a friability of less than 2% when measured by U.S.P. and is adapted to disintegrating spontaneously in the mouth of a patient in less than about 60 seconds (and, more preferably, less than about 30 seconds) and thereby liberate said particles and be capable of being stored in bulk.

In another embodiment the present invention describes a compressed fast disintegrating tablet comprise effervescent agents. Examples of effervescent pharmaceutical compositions suitable for use in conjunction with the present invention are the compositions described in Pather, U.S. Pat. No. 6,200,604, which is incorporated herein by reference. Other pharmaceutical compositions suitable for use in conjunction with the present invention are the compositions described in U.S. Pat. No. 5,178,878 to Wehling, et al., U.S. Pat. No. 5,223,264 to Wehling, et al. and U.S. Pat. No. 6,024,981 to Khankari et al. which are incorporated herein by reference.

In one embodiment, the present invention describes a dosage form as a fast disintegrating ordered-mixture composition as disclosed in European patent EP 0 324 725 (herein incorporated by reference). In these compositions clozapine, and/or a metabolite of clozapine, covers (in a finely dispersed state) the surface of substantially larger carrier particles.

Such compositions disintegrate rapidly in water, thereby dispersing their contents of microscopic drug particles.

Similarly, substantial weight gain is also associated with the use of some antidepressants. While it is not intended the present invention be limited to any specific mechanism, proposed mechanisms which account for weight gain secondary to the administration of, for example, tricyclic antidepressants include noradrenergic or antihistaminic inhibition of satiety and decreased metabolic rate. In contrast, proposed mechanisms which account for weight gain secondary to the administration of monoamine oxidase include insulin-like actions on carbohydrate and fat metabolism, polydipsia, and sodium retention. While it is not intended the present invention be limited to any specific mechanism, the Applicant believes the fast disintegrating formulations described by embodiments of the present invention present antidepressants to the gastrointestinal tract (hereinafter referred to as the “GI tract”) under conditions such that normal satiety feedback is not disrupted. As noted above, satiety signals originate in the GI tract. The Applicant believes that conventional formulations of antidepressants (selected examples include but are not limited to: phenelzine (NARDIL), tranylcypromine (PARNATE), amitriptyline (ELAVIL, ENDEP), Bupropion (WELLBUTRIN), Fluoxetine (PROZAC), Mirtazapine (REMERON), Nefazadone (SERZONE), Paroxetine (PAXIL), and Sertraline (ZOLOFT) present antidepressants to the stomach lining as a bolus of drug which interacts with satiety receptors in the stomach under conditions such that a patient continues to eat without feeling a sense of fullness. The extra caloric intake associated with this “over eating” promotes weight gain in patients taking these conventional formulations of antidepressants.

In contrast, the fast disintegrating formulations described by embodiments of the instant invention present antidepressants to the stomach in a metered fashion. That is to say the fast disintegrating formulations of antidepressants, described by embodiments of the present invention, are converted into a slurry when contacted with the saliva in the mouth. This drug and saliva slurry is delivered to the stomach via the reflexive swallowing of the saliva. The Applicant believes this change, as compared to conventional formulations of atypical antipsychotics, in the way the fast disintegrating formulations of the present invention are delivered to the stomach prevents (or reduces) interference with satiety receptors in the stomach. A patient taking any of the fast disintegrating formulations described by embodiments of the present invention will experiences a sense of fullness upon completing a meal and, therefore, is less likely to experience weight gain as a function of overeating. It is not intended that the present invention be limited to any specific dose of antidepressant.

In one embodiment, the present invention comprises treating a patient suffering from one or more symptoms of depression with an antidepressant that does not promote weight gain in a patient with a substantially normal BMI. It is contemplated that oral administration of antidepressants may be made with a fast disintegrating formulation. In one embodiment this fast disintegrating formulation is an ODT.

In one embodiment the present invention describes a hard, compressed, fast disintegrating tablet, formulated with an antidepressant, adapted for oral administration. The tablet includes particles made of an active ingredient and a protective material. These particles are provided in an amount of between about 0:01 and about 75% by weight based on the weight of the tablet. The tablet may also include a matrix made from a nondirect compression filler, a wicking agent, and a hydrophobic lubricant. The preferred tablet matrix comprises at least about 60% rapidly water-soluble ingredients based on the total weight of the matrix material. The preferred tablet has a hardness of between about 15 and about 50 Newtons, a friability of less than 2% when measured by U.S.P. and is adapted to disintegrating spontaneously in the mouth of a patient in less than about 60 seconds (and, more preferably, less than about 30 seconds) and thereby liberate said particles and be capable of being stored in bulk.

In another embodiment the present invention describes a compressed fast disintegrating tablet, formulated with an antidepressant, that also comprises one or more effervescent agents. Examples of effervescent pharmaceutical compositions suitable for use in conjunction with the present invention are the compositions described in Pather, U.S. Pat. No. 6,200,604, which is incorporated herein by reference.

In one embodiment, the present invention describes a dosage form as a fast disintegrating ordered-mixture composition as disclosed in European patent EP 0 324 725 (herein incorporated by reference). In these compositions an antidepressant, in a finely dispersed state, covers the surface of substantially larger carrier particles. Such compositions disintegrate rapidly in water, thereby dispersing their contents of microscopic drug particles. In one embodiment the present invention describes a method for reducing body weight comprising providing: i) a patient having a BMI greater than 25 demonstrating at least one symptom of psychosis and, ii) a fast disintegrating formulation of an atypical antipsychotic and; administering said fast disintegrating formulation of an atypical antipsychotic under conditions such that said BMI is reduced. In a preferred embodiment said atypical antipsychotic is clozapine. In an especially preferred embodiment said fast disintegrating formulation of an atypical antipsychotic is FAZACLO.

In one embodiment the present invention describes a method for reducing body weight comprising: providing a patient who has been taking a conventional formulation of an atypical antipsychotic for a period of greater than three weeks; measuring said patient so as to record a first body weight measurement; substituting a fast disintegrating formulation of an atypical antipsychotic in place of the conventional formulation of an atypical antipsychotic taken by said patient; measuring said patient at an interval after said substitution so as to record a second body weight measurement wherein said second body weight measurement is less that said first body weight measurement, thereby, confirming a reduction in weight. In one embodiment, said atypical antipsychotic is clozapine. In a preferred embodiment said fast disintegrating clozapine formulation is FAZACLO.

In one embodiment, the present invention describes the method, set out in the paragraph above, wherein said interval of time is in a range between one and twelve weeks and in another embodiment, said interval of time is in a range between two and four weeks. In one embodiment, the present invention describes a method for reducing body weight comprising providing a patient treated with a conventional formulation of an atypical antipsychotic and; treating said patient with a fast disintegrating formulation selected from the group consisting of dibenzodiazepines, pyridopyrimidinones, dibenzothiazepines, halodihydroindolones and, tetrahydroquinolinones under conditions such that body weight is reduced. In one embodiment said fast disintegrating dibenzodiazapine is clozapine. In a preferred embodiment, said fast disintegrating clozapine formulation is FAZACLO.

In one embodiment the present invention describes a method for reducing body weight comprising providing a patient treated with a conventional formulation of a dibenzodiazapine and treating said patient with a fast disintegrating formulation of a dibenzodiazapine under conditions such that body weight is reduced. In one embodiment said fast disintegrating formulation of a dibenzodiazapine is clozapine. In a preferred embodiment, said fast disintegrating clozapine formulation is FAZACLO.

In one embodiment the present invention describes a method for reducing body weight comprising providing a patient who has been taking a conventional formulation of an atypical antipsychotic for a period of greater than three weeks; discontinuing administration of said conventional formulation of an atypical antipsychotic and; treating said patient, after said discontinuation of said conventional formulation of an atypical antipsychotic, with a fast disintegrating formulation of a dibenzodiazapine under conditions such that body weight is reduced. In one embodiment, the interval between the discontinuation of said conventional formulation of an atypical antipsychotic and the treatment of said patient with said fast disintegrating formulation of a dibenzodiazapine is in the range between one and three days. In one embodiment said fast disintegrating formulation of a dibenzodiazapine is clozapine. In a preferred embodiment, said fast disintegrating clozapine formulation is FAZACLO.

In one embodiment the present invention describes a method for reducing body weight comprising providing a patient treated with a conventional formulation of an atypical antipsychotic and; treating said patient with a fast disintegrating formulation comprising clozapine and a compound selected from the group consisting of antidepressants, anticonvulsants, and antianxiety drugs under conditions such that body weight is reduced.

In one embodiment the present invention describes a method for reducing body weight comprising providing a patient treated with a conventional formulation of a dibenzodiazapine and treating said patient with a fast disintegrating formulation of a dibenzodiazapine under conditions such that body weight is reduced. In one embodiment said fast disintegrating formulation of a dibenzodiazapine is clozapine. In a preferred embodiment, said fast disintegrating clozapine formulation is FAZACLO.

In one embodiment the present invention describes a method for reducing body weight comprising providing a patient who has been taking a conventional formulation of an atypical antipsychotic for a period of greater than three weeks; discontinuing administration of said conventional formulation of an atypical antipsychotic and; treating said patient, after said discontinuation of said conventional formulation of an atypical antipsychotic, with a fast disintegrating formulation of a dibenzodiazapine under conditions such that body weight is reduced. In one embodiment, the interval between the discontinuation of said conventional formulation of an atypical antipsychotic and the treatment of said patient with said fast disintegrating formulation of a dibenzodiazapine is in the range between one and three days. In one embodiment said fast disintegrating formulation of a dibenzodiazapine is clozapine. In a preferred embodiment, said fast disintegrating clozapine formulation is FAZACLO. In one embodiment the present invention describes a method for reducing body weight comprising providing a patient treated with a conventional formulation of an atypical antipsychotic and; treating said patient with a fast disintegrating formulation comprising clozapine and a compound selected from the group consisting of antidepressants, anticonvulsants, and antianxiety drugs under conditions such that body weight is reduced.

In one embodiment the present invention describes a method for reducing body weight comprising: providing a patient who has been taking a conventional formulation of an atypical antipsychotic for a period of greater than three weeks; measuring said patient so as to record a first body weight measurement; substituting a fast disintegrating formulation comprising a metabolite of clozapine in place of the conventional formulation of an atypical antipsychotic taken by said patient; measuring said patient at an interval after said substitution so as to record a second body weight measurement wherein said second body weight measurement is less that said first body weight measurement, thereby, confirming a reduction in weight. In one embodiment, said metabolite of clozapine is desmethyl clozapine. In a preferred embodiment said fast disintegrating desmethyl clozapine formulation is and ODT.

In one embodiment, the present invention describes the method, set out in the paragraph above, wherein said interval of time is in a range between one and twelve weeks and in another embodiment, said interval of time is in a range between two and four weeks. In one embodiment, the present invention describes a method for reducing body weight comprising providing a patient treated with a conventional formulation of an atypical antipsychotic and; treating said patient with a fast disintegrating formulation selected from the group consisting of compounds 1-14 set out in FIG. 1 under conditions such that body weight is reduced.

In one embodiment the present invention describes a method for reducing body weight comprising: providing a patient who has been taking a conventional formulation of an antidepressant for a period of greater than three weeks; measuring said patient so as to record a first body weight measurement; substituting a fast disintegrating formulation comprising an antidepressant in place of the conventional formulation of an atypical antipsychotic taken by said patient; measuring said patient at an interval after said substitution so as to record a second body weight measurement wherein said second body weight measurement is less that said first body weight measurement, thereby, confirming a reduction in weight. In one embodiment, said antidepressant is paroxetine (PAXIL). In a preferred embodiment said PAXIL formulation is an ODT.

In one embodiment, the present invention describes the method, set out in the paragraph above, wherein said interval of time is in a range between one and twelve weeks and in another embodiment, said interval of time is in a range between two and four weeks. In one embodiment, the present invention describes a method for reducing body weight comprising providing a patient treated with a conventional formulation of an antidepressant and; treating said patient with a fast disintegrating formulation selected from the group antidepressants, set out in Table 1, under conditions such that body weight is reduced.

In one embodiment the present invention describes a method for reducing body weight comprising: providing a patient treated with a conventional formulation of an antidepressant and; treating said patient with a fast disintegrating antidepressant formulation conditions such that body weight is reduced.

In selected embodiment, said fast disintegrating antidepressant is selected from the group consisting of: citalopram, escitalopram oxalate, fluvoxamine maleate, paroxetine, fluoxetine, sertraline, amitriptyline, clomipramine, desipramine, doxepin, imipramine, nortryptyline, protriptyline, trimipramine, maprotiline, bupropion, buspirone, duloxetine, mirtazapine, nefazodone, reboxetine, trazodone, venlafaxine, phenelzine, tranylcypromine, and lithium carbonate.

In one embodiment, the present invention describes a method for reducing body weight comprising: providing a patient who has been taking a conventional formulation of an antidepressant for a period of greater than three weeks; discontinuing administration of said conventional formulation of an antidepressant and; treating said patient, after said discontinuation of said conventional formulation of an antidepressant, with a fast disintegrating formulation of a antidepressant under conditions such that body weight is reduced. In selected embodiment the interval between the discontinuation of said conventional formulation of an antidepressant and the treatment of said patient with said fast disintegrating formulation of a antidepressant is in the range between one and three days.

In another embodiment, said fast disintegrating formulation of an antidepressant is selected from the group consisting of: citalopram, escitalopram oxalate, fluvoxamine maleate, paroxetine, fluoxetine, sertraline, amitriptyline, clomipramine, desipramine, doxepin, imipramine, nortryptyline, protriptyline, trimipramine, maprotiline, bupropion, buspirone, duloxetine, mirtazapine, nefazodone, reboxetine, trazodone, venlafaxine, phenelzine, tranylcypromine, and lithium carbonate.

In one embodiment, the present invention describes a method for reducing body weight comprising: providing a patient treated with a conventional formulation of a dibenzodiazapine and; treating said patient with a fast disintegrating formulation of 8-Chloro-11-(1-piperazinyl)-5H-dibenzo[b,E][1,4]diazepine under conditions such that body weight is reduced.

In another embodiment, the present invention describes a method for reducing body weight comprising: providing a patient who has been taking a conventional formulation of an atypical antipsychotic for a period of greater than three weeks; discontinuing administration of said conventional formulation of an atypical antipsychotic and; treating said patient, after said discontinuation of said conventional formulation of an atypical antipsychotic, with a fast disintegrating formulation of 8-Chloro-11-(1-piperazinyl)-5H-dibenzo[b,E][1,4]diazepine under conditions such that body weight is reduced.

In one embodiment, the interval between the discontinuation of said conventional formulation of an atypical antipsychotic and the treatment of said patient with said fast disintegrating formulation of a 8-Chloro-1-(1-piperazinyl)-5H-dibenzo[b,E][1,4]diazepine is in the range between one and three days.

In one embodiment, the present invention contemplates a method for modulation body weight comprising, providing: i) a patient exhibiting at least one symptom of psychosis having a BMI of 24 or less, and ii) a fast disintegrating formulation of a clozapine metabolite and administering said fast disintegrating clozapine metabolite formulation for a period of at least four weeks under conditions such that after said fast disintegrating clozapine metabolite treatment, at least four weeks in duration, the BMI for said patient is less than 25.

In a preferred embodiment, said fast disintegrating clozapine metabolite is desmethyl clozapine.

In one embodiment, the present invention contemplates a method for modulating body weight comprising, providing: i) a patient exhibiting at least one symptom of depression having a BMI of 24 or less, and ii) a fast disintegrating formulation of an antidepressant and; administering said fast disintegrating antidepressant formulation for a period of at least four weeks under conditions such that after said fast disintegrating antidepressant treatment, at least four weeks in duration, the BMI for said patient is less than 25. In a preferred embodiment, said fast disintegrating antidepressant is paroxetine.

### DESCRIPTION OF THE FIGURES

FIG. 1 shows a schematic of the metabolites of clozapine wherein “CZ” is an abbreviation for “clozapine”. Compounds 1-14 represent metabolites of clozapine.

### DEFINITIONS

As used herein the term “antipsychotic” refers all drugs used to treat psychosis. Common conditions for which antipsychotics are prescribed include schizophrenia, mania and delusional disorder, although antipsychotics are also used to counter psychosis associated with a wide range of other diagnoses. Antipsychotics also act as mood stabilizers making them suitable for the treatment of bipolar disorder (even when no symptoms of psychosis are present).

As used herein the term “atypical antipsychotics” refer to a class of drugs, used to treat psychosis, which are chemically distinct from “first generation” antipsychotic drugs which include, but are not limited to, phenothiazines, thioxanthines, butyrophenones, diphenylbutylpiperadines and the indolones. Atypical antipsychotic include, but are not limited to, dibenzodiazepines, pyridopyrimidinones, dibenzothiazepines, halodihydroindolones and, tetrahydroquinolinones. Functionally, atypical antipsychotics are less likely to cause extra-pyramidal side effects, drug induced involuntary movements, than “first generation” antipsychotic drugs. Examples of atypical antipsychotic drugs include, but are not limited to, clozapine, risperidone, and quetiapine.

As used herein the term “symptoms of psychosis” includes (but is not limited to) hallucinations, delusions, paranoia, mania, depression, emotional changes, personality changes, behavioral changes, and lack of awareness of mental changes.

As used herein the term “oral administration” is defined as a mode of administration of a pharmaceutical in which the pharmaceutical compound is administered by mouth.

As used herein the term “therapeutic formulation” shall be described as a pharmaceutical composition comprising at least one active ingredient (in one example an active ingredient is an atypical antipsychotic) along with other optional ingredients useful in, for example, binding, flavoring, coloring, and preserving the formulation.

As used herein the term “active ingredient(s)” refers to that fraction of a therapeutic formulation or dosage form (for example: a capsule, a tablet, or syrup) comprising drug(s) which is pharmaceutically active as opposed to the remaining fraction of the therapeutic formulation or dosage form which comprises the “excipient” which is the substantially pharmaceutically inert material the active ingredient(s) is formulated with.

As used herein the term “dosage form,” in accordance with the present invention, includes, but is not limited to, tablets.

As used herein the term “fast disintegrating” shall be defined as the ingredients which will allow rapid disintegration of a dosage form in 60 seconds or less and most preferably 30 seconds or less.

As used herein the acronym “ODT” refer to an “Orally Disintegrating Tablet”. ODT tablets are particular types of fast disintegrating dosage forms that disintegrate in the oral cavity in 0.5-60 seconds and do not need to be swallowed with water as these dosage forms only require native saliva to disintegrate the dosage form, which is then swallowed.

As used herein the terms “effervescent agent” and “effervescent disintegration agent” shall be defined as compounds that evolve gas. The preferred effervescent agents evolve gas by means of a chemical reaction that takes place upon exposure of the effervescent agent to an aqueous solution such as water or saliva.

As used herein the term “Ordered mixture” shall be defined as, and synonymous with, a homogeneous mixture. In the context of the present invention, a homogeneous mixture is a mixture in which the constituents are evenly dispersed or nearly evenly dispersed (e.g., 90% dispersed).

As used herein the word “disintegrant” shall be defined as a component of a solid formulation that acts as a agent that promotes the fragmentation or breakdown of the formulation.

As used herein the word “patient” shall be defined as a person having any symptom of psychosis.

As used herein the acronym “BMI” refers to “Body Mass Index”: an index that relates body weight to height. The body mass index (BMI) is obtained by dividing a person's weight in kilograms (kg) by their height in meters (m) squared. The National Institutes of Health defines normal weight, overweight, and obesity according to the BMI. Since the BMI describes the body weight relative to height, it correlates strongly (in adults) with the total body fat content. A normal BMI is between 20 and 25. Overweight is defined as a BMI between 25 and 30%. Obesity is defined as a BMI over 30.

As used herein the term “conventional formulations of atypical antipsychotics” refer to non-rapidly disintegrating formulations. Examples of a conventional formulation of an atypical antipsychotic include, but are not limited to, CLOZARIL (Novartis) and Clozapine Tablets (Mylan).

As used herein the term “conventional formulations of antidepressants” refer to non-rapidly disintegrating formulations. Examples of a conventional formulation of antidepressants include, but are not limited to: CELEXA, CIPRAMIL, CIPRALEX, LEXAPRO LUVOX, PAXIL, SEROXAT, AROPAX, PROZAC, ZOLOFT, LUSTRAL, NARDIL, PARNATE, ELAVIL, ENDEP, ANAFRANIL, NORPRAMIN, PERTOFRANE, ADAPIN, SINEQUAN, TOFRANIL, PAMELOR, VIVACTIL, SURMONTIL, LUDIOMIL, WELLBUTRIN), BUSPAR, CYMBALTA, REMERON, ZISPIN, AVANZA, NORSET, REMERGIL, SERZONE, EDRONAX, VESTRA, DESYREL, and EFFEXOR.

As used herein the word “antidepressant” refers to compounds which are administered to reduce the symptoms of clinical depression. Examples of antidepressants include, but are not limited to; i) the SSRIs or selective serotonin reuptake inhibitors. SSRIs include, but are not limited to, citalopram, escitalopram oxalate, fluvoxamine maleate, paroxetine, fluoxetine, sertraline; ii) the MAOIs or monoamine oxidase inhibitors. MAOIs include, but are not limited to, phenelzine, tranylcypromine. Tricyclic antidepressants; iii) tricyclic antidepressants include, but are not limited to, amitriptyline, clomipramine, desipramine, doxepin, imipramine, nortryptyline, protriptyline, trimipramine; iv) the tetracyclic antidepressants. Tetracyclic antidepressants include, but are not limited to, maprotiline. Other antidepressants contemplated in embodiments of the present invention include: bupropion, buspirone, duloxetine, mirtazapine, nefazodone, reboxetine, trazodone, venlafaxine, and lithium carbonate.

As used herein the phrase, “symptoms of clinical depression” include, but are not limited to, feelings of overwhelming sadness or fear (or seeming inability to feel emotion) marked decrease of interest in pleasurable activities, disturbed sleep patterns (either insomnia or sleeping more than normal), decrease in activity levels, restless or moving significantly slower than normal, fatigue, both mental and physical, feelings of guilt, lowered self-esteem, decreased ability to concentrate or make decisions and, thinking about death or suicide.

As used herein the word “anticonvulsant” refers to a medication used to control and/or prevent seizures (also referred to as convulsions) or that stop an ongoing series of seizures. Examples of anticonvulsants include, but not limited to, phenobarbital, phenyloin (DILANTIN), carbamazepine, ethosuximide (ZARONTIN), clonazepam (KLONOPIN), midazolam (VERSED), topirimate (QUETIAPNE), gabapentin (NEURONTIN) and, zonisamide (ZONEGRAN).

As used herein the term “antianxiety drug” refers to a tranquilizer used to reduce anxiety, tension and irritability. Examples of antianxiety drugs include, but are not limited to, diazepam (VALIUM), lorazepam (ATIVAN), Alprazolam (XANAX), Chlordiazepoxide (LIBRIUM), Diazepam (VALIUM), Lorazepam (ATIVAN) and, Buspirone (BUSPAR).

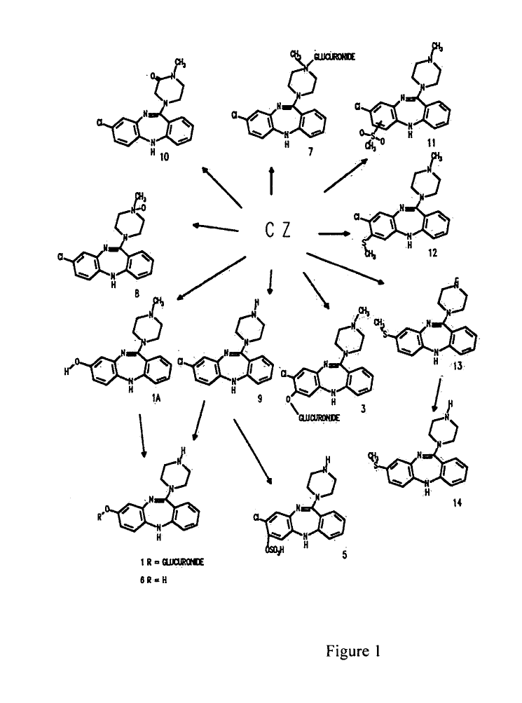

As used herein the term “the metabolites of clozapine” include the group of compounds set out in FIG. 1.

As used herein the term desmethyl clozapine refers to the compound having the IUPAC name of: 8-Chloro-11-(1-piperazinyl)-5H-dibenzo[b,E][1,4]diazepine and the following chemical structure:

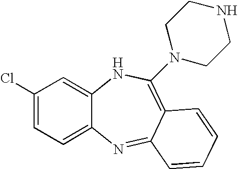

### GENERAL DESCRIPTION OF INVENTION

The present invention describes the administration of atypical antipsychotics, the metabolites of atypical antipsychotics, and antidepressant formulations that reduce body weight in patients who are overweight or obese secondary to treatment with certain psychotropic drugs.

Embodiments of the present invention also describe weight loss in patients previously treated with atypical antipsychotics, including but not limited to formulation such as CLOZARIL (Novartis) and Clozapine Tablets (Mylan), who are switched to the clozapine formulations described in various embodiments of the present invention. The rapidly disintegrating formulations described in various embodiment of the present invention exert an antipsychotic effect, with a reduced incidence of the co-morbidity of weight gain associated with the administration of conventional formulations of clozapine.

Psychosis is a generic psychiatric term for mental states in which the components of rational thought and perception are severely impaired. Persons experiencing a psychosis may experience hallucinations, hold paranoid or delusional beliefs, demonstrate personality changes and exhibit disorganized thinking. These symptoms are usually accompanied by features such as a lack of insight into the unusual or bizarre nature of their behavior, difficulties with social interaction and impairments in carrying out the activities of daily living. Essentially, a psychotic episode involves loss of contact with reality, sometimes termed “loss of reality testing.”

A psychotic episode can be significantly colored by mood. For example, people experiencing a psychotic episode in the context of depression may experience persecutory or self-blaming delusions or hallucinations, while people experiencing a psychotic episode in the context of mania may form grandiose delusions or have an experience of deep religious significance.

Hallucinations are defined as sensory perception in the absence of external stimuli. Psychotic hallucinations may occur in any of the five senses and take on almost any form, which may include simple sensations (such as lights, colors, tastes, smells) to more meaningful experiences such as seeing and interacting with fully formed animals and people, hearing voices and complex tactile sensations. Auditory hallucinations, particularly the experience of hearing voices, are a common and often prominent feature of psychosis. Hallucinated voices may talk about, or to the person, and may involve several speakers with distinct personas. Auditory hallucinations tend to be particularly distressing when they are derogatory, commanding or preoccupying.

Psychosis may involve delusional or paranoid beliefs. Psychotic delusions may be classified into primary and secondary types. Primary delusions are defined as arising out-of-the-blue and not being comprehensible in terms of normal mental processes, whereas secondary delusions may be understood as being influenced by the person's background or current situation.

Thought disorder describes an underlying disturbance to conscious thought and is classified largely by its effects on speech and writing. Affected persons may show pressure of speech (speaking incessantly and quickly), derailment or flight of ideas (switching topic mid-sentence or inappropriately), thought blocking, rhyming or punning. In addition, affected persons may also demonstrate illogical thinking and an inability to abstract.

Psychosis is usually accompanied by a lack of insight into the unusual, strange or bizarre nature of the person's experience or behavior. Even in the case of an acute psychosis, sufferers may seem completely unaware that their vivid hallucinations and impossible delusions are in any way unrealistic. This is not an absolute; however, insight can vary between individuals and throughout the duration of the psychotic episode.

The etiology of psychosis is varied. Psychosis may be the result of an underlying mental illness such as bipolar disorder (also known as manic depression), and schizophrenia. Psychosis may also be triggered or exacerbated by severe mental stress and high doses or chronic use of drugs such as amphetamines, LSD, PCP, cocaine or scopolamine. Sudden withdrawal from CNS depressant drugs, such as alcohol and benzodiazepines, may also trigger psychotic episodes. As can be seen from the wide variety of illnesses and conditions in which psychosis has been reported to arise (including for example, AIDS, leprosy, malaria and even mumps) there is no singular cause of a psychotic episode.

Psychosis has been divided into two major categories: i) depressive insanity (now called bipolar disorder) and ii) dementia praecox (now called schizophrenia).

Psychotic episodes may vary in duration between individuals. In brief reactive psychosis, the psychotic episode is related directly to a specific stressful life event, so patients may spontaneously recover normal functioning within two weeks. In some rare cases, individuals may remain in a state of full-blown psychosis for many years, or perhaps have attenuated psychotic symptoms (such as low intensity hallucinations) present at most times.

Management of psychosis is complicated by the fact that may of the drugs most effective in reducing symptoms of psychosis are associated with significant co-morbidities. Specifically, the administration of atypical antipsychotics are associated with gains in body weight that may increase a patient's BMI to an overweight or obese level. Thus, there is a need for formulations of atypical antipsychotics (already known to be safe and effective in the treatment of psychosis) that do not contribute to one of the most deleterious co-morbidities associated with the administration of the same: weight gain to the point of obesity.

Similarly, “depression” refers to an illness that involves the body, mood, and thoughts. It affects the way a person eats and sleeps, the way one feels about oneself, and the way one thinks about things. Depression, also referred to as a depressive disorder, is not the same as a passing “blue mood”. Without treatment, symptoms can last for weeks, months, or years.

Depressive disorders come in different forms. “Major depression” is manifested by a combination of symptoms that interfere with the ability to work, study, sleep, eat, and enjoy once pleasurable activities. Such a disabling episode of depression may occur only once but more commonly occurs several times in a lifetime.

A less severe type of depression, “dysthymia”, involves long-term, chronic symptoms that do not disable, but keep can impact interpersonal interactions and feeling of well being.

Another type of depression is bipolar disorder, also called manic-depressive illness. Not nearly as prevalent as other forms of depressive disorders, bipolar disorder is characterized by cycling mood changes: severe highs (mania) and lows (depression). Sometimes the mood switches are dramatic and rapid, but most often they are gradual. When in the depressed cycle, an individual can have any or all of the symptoms of a depressive disorder. When in the manic cycle, the individual may be overactive, over talkative, and have a great deal of energy. Mania often affects thinking, judgment, and social behavior in ways that cause serious problems and embarrassment. For example, the individual in a manic phase may feel elated, full of grand schemes that might range from unwise business decisions to romantic sprees. Mania, left untreated, may evolve into psychotic state which is clinically distinct from depression.

Not everyone who is depressed or manic experiences every symptom. Severity of symptoms varies with individuals and also may vary over time. Symptoms of depression include (but are not limited to): persistent sad, anxious, or “empty” mood; feelings of hopelessness, pessimism; feelings of guilt, worthlessness, helplessness; loss of interest or pleasure in hobbies and activities that were once enjoyed, including sex; decreased energy, fatigue, being “slowed down”; difficulty concentrating, remembering, or making decisions; insomnia, early-morning awakening, or oversleeping; appetite and/or weight loss or overeating and weight gain; thoughts of death or suicide; suicide attempts, restlessness, irritability; persistent physical symptoms that do not respond to treatment, such as headaches, digestive disorders, and chronic pain.

In contrast, mania presents with a constellation of symptoms that include, but are not limited to, abnormal or excessive elation; unusual irritability; decreased need for sleep; grandiose notions; increased talking; racing thoughts; increased sexual desire; markedly increased energy; poor judgment; and inappropriate social behavior.

Management of depression is also complicated by the fact that may of the drugs most effective in reducing symptoms of depression are associated with significant co-morbidities. Specifically, the administration of many tricyclic antidepressants are associated with gains in body weight that may increase a patient's BMI to an overweight or obese level. Thus, there is a need for formulations of antidepressants (already known to be safe and effective in the treatment of psychosis) that do not contribute to one of the most deleterious co-morbidities associated with the administration of the same: weight gain to the point of obesity.

### DETAILED DESCRIPTION OF PREFERRED EMBODIMENTS

I. Formulations Comprising Atypical Antipsychotics

In selected embodiments, the present invention is directed to rapidly disintegrating formulations of atypical antipsychotics that do not result in weight gain as a side effect or which minimize the degree of weight gain, over time, as compared to conventional formulations of atypical antipsychotic drugs. In addition administration of these same rapidly disintegrating formulations of atypical antipsychotics, in overweight or obese patients previously taking conventional atypical antipsychotics, is associated with significant weight loss.

The rapidly disintegrating formulations of atypical antipsychotics described by selected embodiment of the present invention are administered orally. Although the present invention is not limited to any particular mechanism, it is believed these rapidly ODT formulations present the drug to the stomach lining without substantially interfering with satiety receptors and/or suppressing insulin release, promoting insulin resistance, or impairing of glucose utilization. This allows these ODT formulations of atypical antipsychotics to exert their antipsychotic effects without promoting significant weight gain. In one embodiment the ODT formulation of an atypical antipsychotic is clozapine. In a preferred embodiment the rapidly disintegrating formulation of clozapine is the ODT formulation having the trade name FAZACLO (Alamo Pharmaceuticals, LLC, Beverly Hills, Calif.).

FAZACLO is a tablet form of clozapine that incorporates the proprietary ORASOLV and DURASOLV technologies licensed from Cima Labs, Inc. (Minneapolis, Minn.). FAZACLO rapidly disintegrates in the mouth with a pleasant mint flavor and is then swallowed reflexively in saliva. FAZACLO is convenient to take as no water is required for administration. Specifically, FAZACLO is a compressed powder tablet designed to disintegrate in the mouth in approximately 5 to 30 seconds as disclosed in U.S. Pat. Nos. 5,178,878, 6,024,981 and 6,221,392, incorporated herein by reference.

FAZACLO's ease of administration is desirable in the treatment of psychotics. That is to say, given that FAZACLO need not be swallowed intact, the dosage form is less likely to be cheeked or pouched by a patient undergoing therapy for the treatment of psychosis.

In another embodiment, the formulation of the tablet has a low grit component for a pleasant mouth feel. The active component of the tablet can be coated within a protective material if required for taste masking. In this regard, the present invention relates to a compressed, rapidly disintegrating dosage form adapted for oral dosing. The dosage form includes clozapine and a mix of inert ingredients. The mix is composed of at least a non-direct compression filler and a lubricant. The dosage form is adapted to rapidly disintegrating in the mouth of a patient without the need for water. Preferably, the dosage form has a friability of about 2% or less when tested according to the U.S.P. The dosage form also preferably has a hardness of 15-50 Newtons (N). The dosage forms described above are able to disintegrating rapidly in the mouth of the patient, with a minimum of grit. The protective materials used in some embodiments of the present invention may include any of the polymers conventionally utilized in the formation of micro particles, matrix-type micro particles and microcapsules. Among these are cellulosic materials such as naturally occurring cellulose and synthetic cellulose derivatives; acrylic polymers and vinyl polymers. Other simple polymers include proteinaceous materials such as gelatin, polypeptides and natural and synthetic shellacs and waxes. Protective polymers may also include ethylcellulose, methylcellulose, carboxymethyl cellulose and acrylic resin material sold under the registered trademark EUDRAGIT by Rhone Pharma GmbH of Weiterstadt, Germany.

In another embodiment, an ODT formulation of an atypical antipsychotic includes an effervescent agent. The effervescent agent is provided in an amount of between about 5% and about 95% by weight, based on the weight of the finished tablet, and more preferably in an amount of between about 30% and about 80% by weight. It is particularly preferred that sufficient effervescent material be provided such that the evolved gas is more than about 5 cm but less that about 30 cm, upon exposure of the tablet to an aqueous environment. Compositions, suitable for oral administration, comprising effervescent agents are provided in U.S. Pat. No. 6,200,604 which is incorporated herein by reference.

In one embodiment, the effervescent agent(s) of the present invention evolve carbon dioxide. Although not limited to a particular mechanism, this reaction is most often the result of the reaction of a soluble acid source and a source of carbon dioxide such as an alkaline carbonate or bicarbonate. The effervescent agent(s) of the present invention is not always based upon a reaction which forms carbon dioxide. The acid sources may be any which are safe for human consumption and may generally include food acids, acid and hydrite antacids such as, for example: citric acid, tartaric, amalic, fumeric, adipic and succinics. Carbonate sources include (but are not limited to) dry solid carbonate and bicarbonate salt such as, preferably, sodium bicarbonate, sodium carbonate, potassium bicarbonate and potassium carbonate, magnesium carbonate and the like. Reactants which evolve oxygen or other gasses which are safe for human consumption are also considered within the scope of the present invention.

In addition to the effervescence-producing agents described above, the rapidly disintegrating formulations of atypical antipsychotics described in embodiment of the present invention may also include suitable non-effervescent disintegration agents. These non-effervescent disintegration agents include (but are not limited to) microcrystalline, cellulose, croscarmelose sodium, crospovidone, starches, corn starch, potato starch and modified starches thereof, sweeteners, clays, such as bentonite, alginates, gums such as agar, guar, locust bean, karaya, pecitin and tragacanth. In preferred embodiments, disintegrants may comprise up to about 20 weight percent and preferably between about 2 and about 10% of the total weight of the composition.

In some embodiments the dosage forms of the present invention may also include glidants, lubricants, binders, sweeteners, flavoring and coloring components. Any conventional sweetener or flavoring component may be used. Combinations of sweeteners, flavoring components, or sweeteners and flavoring components may likewise be used.

Examples of binders which can be used include acacia, tragacanth, gelatin, starch, cellulose materials such as methyl cellulose and sodium carboxy methyl cellulose, alginic acids and salts thereof, magnesium aluminum silicate, polyethylene glycol, guar gum, polysaccharide acids, bentonites, sugars, invert sugars and the like. In preferred embodiments, binders may be used in an amount of up to 60 weight percent and preferably about 10 to about 40 weight percent of the total composition.

Coloring agents may include titanium dioxide, and dyes suitable for food such as those known as F.D.& C. dyes and natural coloring agents such as grape skin extract, beet red powder, beta-carotene, annato, carmine, turmeric, paprika, etc. In preferred embodiments, the amount of coloring used may range from about 0.1 to about 3.5 weight percent of the total composition.

Flavors may be incorporated into the dosage forms of the present invention. These flavors include, but are not limited to, synthetic flavor oils and flavoring aromatics and/or natural oils, extracts from plants, leaves, flowers, fruits and combinations thereof. These may include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. In selected embodiments, flavors may be present in an amount ranging from about 0.05 to about 3 percent by weight based upon the weight of the dosage form.

In preferred embodiments, the present invention contemplates the administration of a solid, oral tablet dosage form suitable for oral administration. Excipient fillers can be used to facilitate tabletting. Theses fillers can also assist in the rapid disintegration of the dosage form in the mouth. Examples of suitable fillers include (but are not limited to) mannitol, dextrose, lactose, sucrose, and calcium carbonate.

In some embodiments of the present invention, atypical antipsychotics are co-formulated with other active ingredients into a fast disintegrating dosage form. In selected embodiments said “other active ingredients” are selected from the group consisting of antidepressants, anticonvulsants, and antianxiety drugs.

In another embodiment of the present invention, atypical antipsychotics are co-formulated with antiparkinson drugs (i.e. cogentin) and other antipsychotics (i.e. seroquel) into a fast disintegrating dosage form.

II. Formulations Comprising the Metabolites of Antipsychotics

In other embodiments, the present invention is directed to rapidly disintegrating formulations of the metabolites of antipsychotic drugs that do not result in weight gain as a side effect or which minimize the degree of weight gain, over time, as compared to conventional formulations of atypical antipsychotic drugs. In addition administration of these same rapidly disintegrating formulations of metabolites of antipsychotic drugs, in overweight or obese patients previously taking conventional metabolites atypical drugs, is associated with significant weight loss.

In another embodiment, the present invention contemplates the administration of fast disintegrating metabolites of antipsychotic drugs to patient demonstrating at least one symptom of psychosis with a baseline BMI of 24 or less, under conditions such that after treatment, for a period of at least four weeks, the BMI for said patient is less than 25. In a preferred embodiment, said metabolite of an antipsychotic drug is desmethyl clozapine.

The rapidly disintegrating formulations of the metabolites of antipsychotic drugs described by selected embodiment of the present invention are administered orally. Although the present invention is not limited to any particular mechanism, it is believed these rapidly ODT formulations present the drug to the stomach lining without substantially interfering with satiety receptors and/or suppressing insulin release, promoting insulin resistance, or impairing of glucose utilization. This allows these ODT formulations of the metabolites of antipsychotic drugs to exert their antipsychotic effects without promoting significant weight gain. In one embodiment the ODT formulation of an antipsychotic drug metabolite is desmethyl clozapine.

In one embodiment this ODT desmethyl clozapine will incorporate the proprietary ORASOLV and DURASOLV technologies licensed from Cima Labs, Inc. (Minneapolis, Minn.). Such a formulation will appear as a compressed powder tablet designed to disintegrate in the mouth in approximately 5 to 30 seconds as disclosed in U.S. Pat. Nos. 5,178,878, 6,024,981 and 6,221,392, incorporated herein by reference.

Such an ODT's ease of administration is desirable in the treatment people demonstrating one or more symptoms of depression. That is to say, given this type of ODT need not be swallowed intact, the dosage form is less likely to be cheeked or pouched by a patient undergoing therapy for the treatment of depression.

In another embodiment, the formulation of the tablet has a low grit component for a pleasant mouth feel. The active component of the tablet can be coated within a protective material if required for taste masking. In this regard, the present invention relates to a compressed, rapidly disintegrating dosage form adapted for oral dosing. The dosage form includes desmethyl clozapine and a mix of inert ingredients. The mix is composed of at least a non-direct compression filler and a lubricant. The dosage form is adapted to rapidly disintegrating in the mouth of a patient without the need for water. Preferably, the dosage form has a friability of about 2% or less when tested according to the U.S.P. The dosage form also preferably has a hardness of 15-50 Newtons (N). The dosage forms described above are able to disintegrating rapidly in the mouth of the patient, with a minimum of grit.

The protective materials used in some embodiments of the present invention may include any of the polymers conventionally utilized in the formation of micro particles, matrix-type micro particles and microcapsules. Among these are cellulosic materials such as naturally occurring cellulose and synthetic cellulose derivatives; acrylic polymers and vinyl polymers. Other simple polymers include proteinaceous materials such as gelatin, polypeptides and natural and synthetic shellacs and waxes. Protective polymers may also include ethylcellulose, methylcellulose, carboxymethyl cellulose and acrylic resin material sold under the registered trademark EUDRAGIT by Rhone Pharma GmbH of Weiterstadt, Germany.

In another embodiment, an ODT formulation comprising a metabolite of an antipsychotic drugs also includes an effervescent agent. The effervescent agent is provided in an amount of between about 5% and about 95% by weight, based on the weight of the finished tablet, and more preferably in an amount of between about 30% and about 80% by weight. It is particularly preferred that sufficient effervescent material be provided such that the evolved gas is more than about 5 cm but less that about 30 cm, upon exposure of the tablet to an aqueous environment. Compositions, suitable for oral administration, comprising effervescent agents are provided in U.S. Pat. No. 6,200,604 which is incorporated herein by reference.

In one embodiment, the effervescent agent(s) of the present invention evolve carbon dioxide. Although not limited to a particular mechanism, this reaction is most often the result of the reaction of a soluble acid source and a source of carbon dioxide such as an alkaline carbonate or bicarbonate. The effervescent agent(s) of the present invention is not always based upon a reaction which forms carbon dioxide. The acid sources may be any which are safe for human consumption and may generally include food acids, acid and hydrite antacids such as, for example: citric acid, tartaric, amalic, fumeric, adipic and succinics. Carbonate sources include (but are not limited to) dry solid carbonate and bicarbonate salt such as, preferably, sodium bicarbonate, sodium carbonate, potassium bicarbonate and potassium carbonate, magnesium carbonate and the like. Reactants which evolve oxygen or other gasses which are safe for human consumption are also considered within the scope of the present invention.

In addition to the effervescence-producing agents described above, the rapidly disintegrating formulations comprising a metabolite of an antipsychotic drugs, described in embodiment of the present invention. may also include suitable non-effervescent disintegration agents. These non-effervescent disintegration agents include (but are not limited to) microcrystalline, cellulose, croscarmelose sodium, crospovidone, starches, corn starch, potato starch and modified starches thereof, sweeteners, clays, such as bentonite, alginates, gums such as agar, guar, locust bean, karaya, pecitin and tragacanth. In preferred embodiments, disintegrants may comprise up to about 20 weight percent and preferably between about 2 and about 10% of the total weight of the composition.

In some embodiments the dosage forms of the present invention may also include glidants, lubricants, binders, sweeteners, flavoring and coloring components. Any conventional sweetener or flavoring component may be used. Combinations of sweeteners, flavoring components, or sweeteners and flavoring components may likewise be used.

Examples of binders which can be used include acacia, tragacanth, gelatin, starch, cellulose materials such as methyl cellulose and sodium carboxy methyl cellulose, alginic acids and salts thereof, magnesium aluminum silicate, polyethylene glycol, guar gum, polysaccharide acids, bentonites, sugars, invert sugars and the like. In preferred embodiments, binders may be used in an amount of up to 60 weight percent and preferably about 10 to about 40 weight percent of the total composition.

Coloring agents may include titanium dioxide, and dyes suitable for food such as those known as F.D.& C. dyes and natural coloring agents such as grape skin extract, beet red powder, beta-carotene, annato, carmine, turmeric, paprika, etc. In preferred embodiments, the amount of coloring used may range from about 0.1 to about 3.5 weight percent of the total composition.

Flavors may be incorporated into the dosage forms of the present invention. These flavors include, but are not limited to, synthetic flavor oils and flavoring aromatics and/or natural oils, extracts from plants, leaves, flowers, fruits and combinations thereof. These may include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. In selected embodiments, flavors may be present in an amount ranging from about 0.05 to about 3 percent by weight based upon the weight of the dosage form.

In preferred embodiments, the present invention contemplates the administration of a solid, oral tablet dosage form suitable for oral administration. Excipient fillers can be used to facilitate tabletting. Theses fillers can also assist in the rapid disintegration of the dosage form in the mouth. Examples of suitable fillers include (but are not limited to) mannitol, dextrose, lactose, sucrose, and calcium carbonate.

In some embodiments of the present invention, metabolites of antipsychotic drugs are co-formulated with other active ingredients into a fast disintegrating dosage form. In selected embodiments said “other active ingredients” are selected from the group consisting of antidepressants, anticonvulsants, and antianxiety drugs.

In another embodiment of the present invention, metabolites of antipsychotic drugs are co-formulated with active ingredients selected from the group consisting of antparkinson drugs and antipsychotics into a fast disintegrating dosage form.

III. Formulation Comprising Antidepressants

In selected embodiments, the present invention is directed to rapidly disintegrating formulations of antidepressants that reduce the likelihood of weight gain as a side effect or which minimize the degree of weight gain, over time, as compared to conventional formulations of antidepressants. In addition administration of these same rapidly disintegrating formulations of antidepressants, in overweight or obese patients previously taking conventional formulations of antidepressants, is associated with significant weight loss.

In another embodiment, the present invention contemplates the administration of fast disintegrating antidepressants to patient demonstrating at least one symptom of depression with a baseline BMI of 24 or less, under conditions such that after treatment, for a period of at least four weeks, the BMI for said patient is less than 25. In a preferred embodiment, said antidepressant is paroxetine.

The rapidly disintegrating formulations of antidepressants described by selected embodiment of the present invention are administered orally. Although the present invention is not limited to any particular mechanism, it is believed these rapidly ODT formulations present the drug to the stomach lining without substantially interfering with satiety receptors and/or suppressing insulin release, promoting insulin resistance, or impairing of glucose utilization. This allows these ODT formulations of antidepressants to exert their antipsychotic effects without promoting significant weight gain. In one embodiment the ODT formulation of an antidepressant is paroxetine (PAXIL). In another embodiment, the ODT formulation of an antidepressant is bupropion (WELLBUTRIN).

In selected embodiments, these ODT formulations of paroxetine (PAXIL) and bupropion (WELLBUTRIN) will incorporate the proprietary ORASOLV and DURASOLV technologies licensed from Cima Labs, Inc. (Minneapolis, Minn.). Moreover, these ODT formulations comprising a antidepressant will present as a compressed powder tablet designed to disintegrate in the mouth in approximately 5 to 30 seconds as disclosed in U.S. Pat. Nos. 5,178,878, 6,024,981 and 6,221,392, incorporated herein by reference.

These ODT antidepressant formulations ease of administration is desirable in the treatment of depression. That is to say given the ODT need not be swallowed intact, the dosage form is less likely to be cheeked or pouched by a patient undergoing therapy for the treatment of depression.

In another embodiment, these ODT formulations have a low grit component for a pleasant mouth feel. The active component of the tablet can be coated within a protective material if required for taste masking. In this regard, the present invention relates to a compressed, rapidly disintegrating dosage form adapted for oral dosing. The dosage form includes paroxetine (PAXIL) and bupropion (WELLBUTRIN) and a mix of inert ingredients. The mix is composed of at least a non-direct compression filler and a lubricant. The dosage form is adapted to rapidly disintegrating in the mouth of a patient without the need for water. Preferably, the dosage form has a friability of about 2% or less when tested according to the U.S.P. The dosage form also preferably has a hardness of 15-50 Newtons (N). The dosage forms described above are able to disintegrating rapidly in the mouth of the patient, with a minimum of grit.

The protective materials used in some embodiments of the present invention may include any of the polymers conventionally utilized in the formation of micro particles, matrix-type micro particles and microcapsules. Among these are cellulosic materials such as naturally occurring cellulose and synthetic cellulose derivatives; acrylic polymers and vinyl polymers. Other simple polymers include proteinaceous materials such as gelatin, polypeptides and natural and synthetic shellacs and waxes. Protective polymers may also include ethylcellulose, methylcellulose, carboxymethyl cellulose and acrylic resin material sold under the registered trademark EUDRAGIT by Rhone Pharma GmbH of Weiterstadt, Germany. In another embodiment, an ODT formulation of an antidepressant includes an effervescent agent. The effervescent agent is provided in an amount of between about 5% and about 95% by weight, based on the weight of the finished tablet, and more preferably in an amount of between about 30% and about 80% by weight. It is particularly preferred that sufficient effervescent material be provided such that the evolved gas is more than about 5 cm but less that about 30 cm, upon exposure of the tablet to an aqueous environment. Compositions, suitable for oral administration, comprising effervescent agents are provided in U.S. Pat. No. 6,200,604 which is incorporated herein by reference.

In one embodiment, the effervescent agent(s) of the present invention evolve carbon dioxide. Although not limited to a particular mechanism, this reaction is most often the result of the reaction of a soluble acid source and a source of carbon dioxide such as an alkaline carbonate or bicarbonate. The effervescent agent(s) of the present invention is not always based upon a reaction which forms carbon dioxide. The acid sources may be any which are safe for human consumption and may generally include food acids, acid and hydrite antacids such as, for example: citric acid, tartaric, amalic, fumeric, adipic and succinics. Carbonate sources include (but are not limited to) dry solid carbonate and bicarbonate salt such as, preferably, sodium bicarbonate, sodium carbonate, potassium bicarbonate and potassium carbonate, magnesium carbonate and the like. Reactants which evolve oxygen or other gasses which are safe for human consumption are also considered within the scope of the present invention.

In addition to the effervescence-producing agents described above, the rapidly disintegrating formulations of antidepressants described in embodiment of the present invention may also include suitable non-effervescent disintegration agents. These non-effervescent disintegration agents include (but are not limited to) microcrystalline, cellulose, croscarmelose sodium, crospovidone, starches, corn starch, potato starch and modified starches thereof, sweeteners, clays, such as bentonite, alginates, gums such as agar, guar, locust bean, karaya, pecitin and tragacanth. In preferred embodiments, disintegrants may comprise up to about 20 weight percent and preferably between about 2 and about 10% of the total weight of the composition.

In some embodiments the dosage forms of the present invention may also include glidants, lubricants, binders, sweeteners, flavoring and coloring components. Any conventional sweetener or flavoring component may be used. Combinations of sweeteners, flavoring components, or sweeteners and flavoring components may likewise be used.

Examples of binders which can be used include acacia, tragacanth, gelatin, starch, cellulose materials such as methyl cellulose and sodium carboxy methyl cellulose, alginic acids and salts thereof, magnesium aluminum silicate, polyethylene glycol, guar gum, polysaccharide acids, bentonites, sugars, invert sugars and the like. In preferred embodiments, binders may be used in an amount of up to 60 weight percent and preferably about 10 to about 40 weight percent of the total composition.

Coloring agents may include titanium dioxide, and dyes suitable for food such as those known as F.D.& C. dyes and natural coloring agents such as grape skin extract, beet red powder, beta-carotene, annato, carmine, turmeric, paprika, etc. In preferred embodiments, the amount of coloring used may range from about 0.1 to about 3.5 weight percent of the total composition.

Flavors may be incorporated into the dosage forms of the present invention. These flavors include, but are not limited to, synthetic flavor oils and flavoring aromatics and/or natural oils, extracts from plants, leaves, flowers, fruits and combinations thereof. These may include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. In selected embodiments, flavors may be present in an amount ranging from about 0.05 to about 3 percent by weight based upon the weight of the dosage form.

In preferred embodiments, the present invention contemplates the administration of a solid, oral tablet dosage form suitable for oral administration. Excipient fillers can be used to facilitate tabletting. Theses fillers can also assist in the rapid disintegration of the dosage form in the mouth. Examples of suitable fillers include (but are not limited to) mannitol, dextrose, lactose, sucrose, and calcium carbonate.

In some embodiments of the present invention, antidepressants are co-formulated with other active ingredients into a fast disintegrating dosage form. In selected embodiments said “other active ingredients” are selected from the group consisting of antipsychotics, anticonvulsants, and antianxiety drugs.

In another embodiment of the present invention, antidepressants are co-formulated with active ingredients selected from the group consisting of cogentin and seroquel-into a fast disintegrating dosage form.

### EXPERIMENTAL

The following examples serve to illustrate certain preferred embodiments and aspects of the present invention and are not to be construed as limiting the scope thereof. More specifically, it is not intended the present invention be limited to any specific formulation. More specifically, the total mass and the amount of drug in a particular formulation may adjusted to produce, for example, a tablet with a particular mass, shape, and amount active ingredient.

In the experimental disclosure which follows, the following abbreviations apply: eq (equivalents); M (Molar); uM (micromolar); N (Normal); mol (moles); mmol (millimoles); umol (micromoles); nmol (nanomoles); g (grams); mg (milligrams); ug (micrograms); L (liters); ml (milliliters); ul (microliters); cm (centimeters); mm (millimeters); um (micrometers); nm (nanometers); and ° C. (degrees Centigrade).

### Example 1

Clozapine, 8-chloro-11-(4-methyl-1-piperazinyl)-5H-dibenzo[b,e] [1,4] diazepine, is available from a number of commercial sources. For example, clozapine is sold by the Sigma corporation (Product Number: C 6305). In the alternative, clozapine may be synthesized according to the following protocol.

7.4 grams of 2-amino-4-chlorodiphenylamine-2′-carboxylic acid (4″ methyl)piperazide and 35 ml of phosphoroxychloride are heated up for 3 hours under reflux in the presence of 1.4 ml of N,N-dimethylaniline. Upon concentration of the reaction mixture in vacuo as far as possible, the residue is distributed between benzene and ammonia/ice water. The benzene solution is extracted with dilute acetic acid. The acid extract is clarified with charcoal and treated with concentrated ammonia water to precipitate the alkaline substance, which is dissolved in ether. The ethereal solution is washed with water and dried over sodium sulfate. The residue obtains yields, after recrystallization from ether/petroleum ether 2.9 g (41% of theoretical yield) of 8-chloro-11-(4-methyl-1-piperazinyl)-5H-dibenzo[b,e] [1,4] diazepine in the form of yellow grains of melting point 182° to 184° C. (from acetone/petroleum ether).

### Example 2

This example describes the preparation of a number of fast disintegrating clozapine formulations. In one example, the Applicant contemplates an oral dosage forms formulated as a tablet. The mass of this tablet should be less than about 1.00 g and more preferably less than about 0.80 g. The tablet may include surface markings, cuttings, grooves, letters and or numerals for the purpose of decoration and/or identification.

Preferably, the tablet includes micro particles containing one or more systemically distributable pharmaceutical ingredients, together with an effervescent disintegrating agent. The size of the tablet will be dependent upon the amount of material used.

The term “systemically distributable pharmaceutical ingredient”, as used in examples 4 and 5, is a pharmaceutical ingredient which is conducted from the mouth to the digestive system for absorption through the stomach or intestines and systemic distribution through the bloodstream. The term is not limited to pharmaceutical ingredients which are systemically active or which systemically distribute over time. For the purposes of the instant example, a systemically distributable pharmaceutical ingredient is the atypical antipsychotic clozapine. The amount of clozapine incorporated in each tablet may be selected according to known principles of pharmacy.

The amount of effervescent disintegration agent contemplated by this example ranges from about 5 to about 50% by weight of the final composition (and preferably between about 15 and about 30% by weight thereof). In a more preferred embodiment, the amount of effervescent disintegration agent contemplated by this example ranges from between about 20 and about 25% by weight of the total composition.

More specifically, tablets contemplated by the instant example should contain an amount of effervescent disintegration agent effective to aid in the rapid and complete disintegration of the tablet when orally administered. By “rapid”, it is understood that the tablets are expected to disintegrate in the mouth of a patient in less than 10 minutes, and more desirably, between about 10 seconds and about 3 minutes.

The dosage form according to this example may further include one or more additional adjuvants which can be chosen from those known in the art including flavors, dilutents, colors, binders, filler, compaction vehicles, and non-effervescent disintegrants.

Tablets according to this example can be manufactured by known tabletting procedures. In common tabletting processes, the material which is to be tableted is deposited into a cavity, and one or more punch members are then advanced into the cavity and brought into intimate contact with the material to be pressed, whereupon compressive force is applied. The material is thus forced into conformity with the shape of the punches and the cavity. Hundreds, and even thousands, of tablets per minute can be produced in this fashion.

Materials to be incorporated in the tablets, other than the micro particles and the effervescent disintegration agent, may be pretreated to form granules that readily lend themselves to tabletting. This process is known as granulation. As commonly defined, “granulation” is any process of size enlargement whereby small particles are gathered together into larger, permanent aggregates to yield a free-flowing composition having a consistency suitable for tabletting. Such granulated compositions may have consistency similar to that of dry sand. Granulation may be accomplished by agitation in mixing equipment or by compaction, extrusion or globulation.

Lubricant, as used in examples 2 and 3, means a material which can reduce the friction arising at the interface of the tablet and the die wall during compression and ejection thereof. Lubricants may also serve to prevent sticking to the punch and, to a lesser extent, the die wall as well. The term “antiadherents” is sometimes used to refer specifically to substances which function during ejection. As used in the present disclosure, however, the term “lubricant” is used generically and includes “antiadherents”. Tablet sticking during formation and/or ejection may pose serious production problems such as reduced efficiency, irregularly formed tablets, and non-uniform distribution of intended agents or ingredients to be delivered thereby. These problems are particularly severe with high speed tabletting approaches and methods.

The atypical antipsychotics formulated according to the instant example are processed into micro particles. Each microparticle incorporates the atypical antipsychotic, in this example clozapine, in conjunction with a protective material. The microparticle may be provided as a microcapsule or as a matrix-type microparticle. Microcapsules typically incorporate a discrete mass of the pharmaceutical ingredient surrounded by a discrete, separately observable coating of the protective material. Conversely, in a matrix-type particle, the pharmaceutical ingredient is dissolved, suspended or otherwise dispersed throughout the protective material. The micro particles desirably are between about 75 and 600 microns mean outside diameter, and more preferably between about 150 and about 500 microns. Micro particles above about 200 microns may be used. Thus, it is contemplated the micro particles will be between about 200 mesh and about 30 mesh U.S. standard size, and more preferably between about 100 mesh and about 35 mesh.

Although such prompt release is preferred, the protective material utilized in the microparticle, preferably, should not dissolve instantaneously in water or saliva. That is to say, the microparticle should resist dissolution and release for a period of time sufficient to permit the patient to swallow the released microcapsules as the tablet disintegrates. Micro particles made using any of the polymeric protective materials discussed below will not dissolve instantaneously.

Methods of microencapsulation are described in the Lieberman text: Pharmaceutical Dosage Form: Tablets Volume 1, Second Edition, New York, 1989, at pages 372-376 (hereby, incorporated by reference). One method taught in Lieberman is the technique of phase separation or coacervation which involves processing three mutually immiscible phases, one containing the pharmaceutical ingredient (in this example, clozapine), another containing the protective coating material and a third containing a liquid vehicle used only in the manufacturing phase. The three phases are mixed and the protective material phase deposits by absorption on the pharmaceutical ingredient phase. After this step, the protective material phase is converted to a substantially solid form by cross-linking or by removal of solvent from this phase.

Other common techniques may be used for forming matrix-type micro particles wherein the pharmaceutical ingredient is dispersed in the protective material. For example, the pharmaceutical ingredient and a solution of a polymeric protective material may be sprayed to form droplets and contacted with a gas such as hot air so as to remove the solvent from the droplets. Such a mixture may also be dried to a solid and then comminuted to form the micro particles. Alternatively, the mixture of the pharmaceutical ingredient and polymeric solution may be mixed with an immiscible liquid phase and the solvent may be removed through this phase. The mixing step may include emulsification of the phase bearing the pharmaceutical ingredient and the protective material in the immiscible liquid phase.

The protective material may incorporate polymers such as those conventionally utilized in protective materials for micro particles. A wide variety of polymers are known for this purpose. Any such known polymeric material, utilized heretofore in production of microcapsules and/or matrix-type micro particles may be employed as a protective material in micro particles according to the instant example. Among these are cellulosic materials such as naturally occurring cellulose and synthetic cellulose derivatives; acrylic polymers and vinyl polymers. Other suitable polymers include proteinaceous materials such as gelatin, polypeptides and natural and synthetic shellacs and waxes. Particularly preferred protective material polymers include ethylcellulose, methylcellulose, carboxymethylcellulose and the acrylic resin materials sold under the registered trademark EUDRAGIT by Rhone Pharma GmbH of Weiterstadt, Germany.

Many of the protective material polymers discussed above have substantial resistance to dissolution in water. Such water-insoluble materials can be used to make delayed-release micro particles. Preferably, however, where the protective material incorporates water-insoluble materials of this nature, it also includes other ingredients to promote more rapid release of the pharmaceutical ingredient. Such release promoters include soluble polymers and, in particular, polyfractional alcohols such as mannitol, as well as magnesium oxide. For example, the acrylic material of the type known as EUDRAGIT RL30-D, when used with conventional coingredients such as methylcellulose and magnesium sterate tends to provide a slow release, typically about 50 percent or less after 30 minutes. However, a protective material incorporating the same polymeric material in conjunction with about 2 to about 4, and preferably about 2.7 parts mannitol per part EUDRAGIT material on a solids basis, and also incorporating about 0.05 to about 0.2, and preferably about 0.09 parts magnesium oxide per part EUDRAGIT solids provides a protective material with substantially immediate release properties.

Table 2 sets out ingredients suitable for the manufacture of a clozapine containing microparticle:

TABLE 2

| Ingredient: | Grams Solids/% By Weight |
|---|---|
| EUDRAGIT RL-30-D | 157.5/23.8 |
| Clozapine | 70.0/10.6 |
| Mannitol | 420.0/63.5 |
| Magnesium Oxide | 14.0/2.1 |

The EUDRAGIT material will be furnished by the manufacturer as a dispersion containing 30% solids (polymer) in water. The quantity needed to provide 157.5 grams solids will be placed in a beaker and mixed to form a vortex. The mannitol and clozapine are added and mixing is continued for 10 minutes. After this 10 minute mixing period, the magnesium oxide is added and mixing is continued for another 10 minutes. These mixing steps will take place at room temperature. The resulting mixture will be poured into a tray and dried in an oven at 50° C. under air for one hour. After one hour, the resulting partially dried mixture will be broken into lumps and then dried for an additional hour at 50° C. The dried lumps are then comminuted to micro particles, and screened through an 8 mesh screen. The screened micro particles will be dried for an additional hour at 60° C.

The fraction of the resulting micro particles passing through a 30 mesh screen will be collected. These micro particles will be tableted into an effervescent tablet of about 1.0-2.0 kilo pounds hardness with an effervescent disintegration agent and other ingredients according to the following formulation set out in Table 3:

TABLE 3

| Ingredient: | mg/Tablet |
|---|---|
| Mannitol | 225.0 mg |
| Aspartame | 40.0 mg |
| Cherry Flavor | 6.0 mg |
| Magnesium Stearate | 5.0 mg |
| Silicon Dioxide | 1.0 mg |
| Sodium Bicarbonate | 100.0 mg |
| Citric Acid | 80.0 mg |
| Micro particles | 94.3 mg |

The effervescent tablet will have a dissolution time of less than about one minute. When administered by mouth, it will provide substantially the same bioavailability offered by conventional formulation of clozapine. The forgoing example illustrates one embodiment of the present invention. It is not intended that the present invention be limited to any specific example. For example, the amount of atypical antipsychotic formulated in a given example may be increased or without departing from the spirit and scope of the invention.

### Example 3

This example presents another fast disintegrating formulation, as set out in Table 4, of clozapine. The other constituents of this tablet may be selected from the ingredients described in Example 2 above.

TABLE 4
Ingredients As A Percentage Of Tablet Mass

| | |
|---|---|
| Clozapine | 30.8% |
| Powdered Mannitol | 51.2% |
| Citric Acid | 1.7% |
| Sweetener | 4.6% |
| Glidant | 0.3% |
| Lubricant | 1.5% |
| Wicking Agent | 5.8% |
| Flavor | 3.8% |
| Color | 0.3% |

(Calculated in view of 650 mg total tablet weight)

Tablets will be produced using a direct compression method as follows. All of the material, except the lubricant, will be weighed and blended for a period of between about 30 and about 50 minutes. Thereafter, the lubricant will be added and the mixture will be blended for an additional 5 to 15 minutes. The blend will then be tableted on a conventional 6 or 16 stage rotating tablet press at 25-30 revolutions per minute. Tablets are compressed using an average compression force of approximately 10.27 kN. These tablets are expected to disintegrate in between 20 and 30 seconds, without the use of an effervescent disintegrant.

### Example 4

Desmethyl clozapine, 8-Chloro-11-(1-piperazinyl)-5H-dibenzo[b,E] [1,4]diazepine), is available from a number of commercial sources. For example, desmethyl clozapine is sold by the Sigma corporation (Product Number: D 5676).

This example describes the preparation of a number of fast disintegrating desmethyl clozapine formulations. In one example, the Applicant contemplates an oral dosage forms formulated as a tablet. The mass of this tablet should be less than about 1.00 g and more preferably less than about 0.80 g. The tablet may include surface markings, cuttings, grooves, letters and or numerals for the purpose of decoration and/or identification.

Preferably, the tablet includes micro particles containing one or more systemically distributable pharmaceutical ingredients, together with an effervescent disintegrating agent. The size of the tablet will be dependent upon the amount of material used.

The term “systemically distributable pharmaceutical ingredient”, as used in examples 4 and 5, is a pharmaceutical ingredient which is conducted from the mouth to the digestive system for absorption through the stomach or intestines and systemic distribution through the bloodstream. The term is not limited to pharmaceutical ingredients which are systemically active or which systemically distribute over time. For the purposes of the instant example, a systemically distributable pharmaceutical ingredient is the desmethyl clozapine clozapine. The amount of desmethyl clozapine incorporated in each tablet may be selected according to known principles of pharmacy.

The amount of effervescent disintegration agent contemplated by this example ranges from about 5 to about 50% by weight of the final composition (and preferably between about 15 and about 30% by weight thereof). In a more preferred embodiment, the amount of effervescent disintegration agent contemplated by this example ranges from between about 20 and about 25% by weight of the total composition.

More specifically, tablets contemplated by the instant example should contain an amount of effervescent disintegration agent effective to aid in the rapid and complete disintegration of the tablet when orally administered. By “rapid”, it is understood that the tablets are expected to disintegrate in the mouth of a patient in less than 10 minutes, and more desirably, between about 10 seconds and about 3 minutes.

The dosage form according to this example may further include one or more additional adjuvants which can be chosen from those known in the art including flavors, dilutents, colors, binders, filler, compaction vehicles, and non-effervescent disintegrants.

Tablets according to this example can be manufactured by known tabletting procedures. In common tabletting processes, the material which is to be tableted is deposited into a cavity, and one or more punch members are then advanced into the cavity and brought into intimate contact with the material to be pressed, whereupon compressive force is applied. The material is thus forced into conformity with the shape of the punches and the cavity. Hundreds, and even thousands, of tablets per minute can be produced in this fashion.

Materials to be incorporated in the tablets, other than the micro particles and the effervescent disintegration agent, may be pretreated to form granules that readily lend themselves to tabletting. This process is known as granulation. As commonly defined, “granulation” is any process of size enlargement whereby small particles are gathered together into larger, permanent aggregates to yield a free-flowing composition having a consistency suitable for tabletting. Such granulated compositions may have consistency similar to that of dry sand. Granulation may be accomplished by agitation in mixing equipment or by compaction, extrusion or globulation.

Lubricant, as used in examples 4 and 5, means a material which can reduce the friction arising at the interface of the tablet and the die wall during compression and ejection thereof. Lubricants may also serve to prevent sticking to the punch and, to a lesser extent, the die wall as well. The term “antiadherents” is sometimes used to refer specifically to is substances which function during ejection. As used in the present disclosure, however, the term “lubricant” is used generically and includes “antiadherents”. Tablet sticking during formation and/or ejection may pose serious production problems such as reduced efficiency, irregularly formed tablets, and non-uniform distribution of intended agents or ingredients to be delivered thereby. These problems are particularly severe with high speed tabletting approaches and methods.

The desmethyl clozapine formulated according to the instant example are processed into micro particles. Each microparticle incorporates the desmethyl clozapine in conjunction with a protective material. The microparticle may be provided as a microcapsule or as a matrix-type microparticle. Microcapsules typically incorporate a discrete mass of the pharmaceutical ingredient surrounded by a discrete, separately observable coating of the protective material. Conversely, in a matrix- type particle, the pharmaceutical ingredient is dissolved, suspended or otherwise dispersed throughout the protective material. The micro particles desirably are between about 75 and 600 microns mean outside diameter, and more preferably between about 150 and about 500 microns. Micro particles above about 200 microns may be used. Thus, it is contemplated the micro particles will be between about 200 mesh and about 30 mesh U.S. standard size, and more preferably between about 100 mesh and about 35 mesh.

Although such prompt release is preferred, the protective material utilized in the microparticle, preferably, should not dissolve instantaneously in water or saliva. That is to say, the microparticle should resist dissolution and release for a period of time sufficient to permit the patient to swallow the released microcapsules as the tablet disintegrates. Micro particles made using any of the polymeric protective materials discussed below will not dissolve instantaneously.

Methods of microencapsulation are described in the Lieberman text: Pharmaceutical Dosage Form: Tablets Volume 1, Second Edition, New York, 1989, at pages 372-376 (hereby, incorporated by reference). One method taught in Lieberman is the technique of phase separation or coacervation which involves processing three mutually immiscible phases, one containing the pharmaceutical ingredient (in this example, desmethyl clozapine), another containing the protective coating material and a third containing a liquid vehicle used only in the manufacturing phase. The three phases are mixed and the protective material phase deposits by absorption on the pharmaceutical ingredient phase. After this step, the protective material phase is converted to a substantially solid form by cross-linking or by removal of solvent from this phase.

Other common techniques may be used for forming matrix-type micro particles wherein the pharmaceutical ingredient is dispersed in the protective material. For example, the pharmaceutical ingredient and a solution of a polymeric protective material may be sprayed to form droplets and contacted with a gas such as hot air so as to remove the solvent from the droplets. Such a mixture may also be dried to a solid and then comminuted to form the micro particles. Alternatively, the mixture of the pharmaceutical ingredient and polymeric solution may be mixed with an immiscible liquid phase and the solvent may be removed through this phase. The mixing step may include emulsification of the phase bearing the pharmaceutical ingredient and the protective material in the immiscible liquid phase.

The protective material may incorporate polymers such as those conventionally utilized in protective materials for micro particles. A wide variety of polymers are known for this purpose. Any such known polymeric material, utilized heretofore in production of microcapsules and/or matrix-type micro particles may be employed as a protective material in micro particles according to the instant example. Among these are cellulosic materials such as naturally occurring cellulose and synthetic cellulose derivatives; acrylic polymers and vinyl polymers. Other suitable polymers include proteinaceous materials such as gelatin, polypeptides and natural and synthetic shellacs and waxes. Particularly preferred protective material polymers include ethylcellulose, methylcellulose, carboxymethylcellulose and the acrylic resin materials sold under the registered trademark EUDRAGIT by Rhone Pharma GmbH of Weiterstadt, Germany.

Many of the protective material polymers discussed above have substantial resistance to dissolution in water. Such water-insoluble materials can be used to make delayed-release micro particles. Preferably, however, where the protective material incorporates water-insoluble materials of this nature, it also includes other ingredients to promote more rapid release of the pharmaceutical ingredient. Such release promoters include soluble polymers and, in particular, polyfractional alcohols such as mannitol, as well as magnesium oxide. For example, the acrylic material of the type known as EUDRAGIT RL30-D, when used with conventional coingredients such as methylcellulose and magnesium sterate tends to provide a slow release, typically about 50 percent or less after 30 minutes. However, a protective material incorporating the same polymeric material in conjunction with about 2 to about 4, and preferably about 2.7 parts mannitol per part EUDRAGIT material on a solids basis, and also incorporating about 0.05 to about 0.2, and preferably about 0.09 parts magnesium oxide per part EUDRAGIT solids provides a protective material with substantially immediate release properties.

Table 5 sets out ingredients suitable for the manufacture of a desmethyl clozapine containing microparticle:

TABLE 5

| Ingredient: | Grams Solids/% By Weight |
|---|---|
| EUDRAGIT RL-30-D | 157.5/23.8 |
| Desmethyl clozapine | 140.0/21.2 |
| Mannitol | 350.0/52.9 |
| Magnesium Oxide | 14.0/2.1 |

The EUDRAGIT material will be furnished by the manufacturer as a dispersion containing 30% solids (polymer) in water. The quantity needed to provide 157.5 grams solids will be placed in a beaker and mixed to form a vortex. The mannitol and desmethyl clozapine are added and mixing is continued for 10 minutes. After this 10 minute mixing period, the magnesium oxide is added and mixing is continued for another 10 minutes. These mixing steps will take place at room temperature. The resulting mixture will be poured into a tray and dried in an oven at 50° C. under air for one hour. After one hour, the resulting partially dried mixture will be broken into lumps and then dried for an additional hour at 50° C. The dried lumps are then comminuted to micro particles, and screened through an 8 mesh screen. The screened micro particles will be dried for an additional hour at 60° C.

The fraction of the resulting micro particles passing through a 30 mesh screen will be collected. These micro particles will be tableted into an effervescent tablet of about 1.0-2.0 kilo pounds hardness with an effervescent disintegration agent and other ingredients according to the following formulation set out in Table 6:

TABLE 6

| Ingredient: | mg/Tablet |
|---|---|
| Mannitol | 225.0 mg |
| Aspartame | 40.0 mg |
| Cherry Flavor | 6.0 mg |
| Magnesium Stearate | 5.0 mg |
| Silicon Dioxide | 1.0 mg |
| Sodium Bicarbonate | 100.0 mg |
| Citric Acid | 80.0 mg |
| Micro particles | 94.3 mg |

The effervescent tablet will have a dissolution time of less than about one minute. When administered by mouth, it will provide substantially the same bioavailability offered by conventional formulation of clozapine. The forgoing example illustrates one embodiment of the present invention. It is not intended that the present invention be limited to any specific example. For example, the amount of desmethyl clozapine formulated in a given example may be increased or without departing from the spirit and scope of the invention.

### Example 5

This example presents another fast disintegrating formulation, as set out in Table 6, of desmethyl clozapine. The other constituents of this tablet may be selected from the ingredients described in Example 5 above.

TABLE 6
Ingredients As A Percentage Of Tablet Mass

| | |
|---|---|
| Desmethyl clozapine | 40.8% |
| Powdered Mannitol | 41.2% |
| Citric Acid | 1.7% |
| Sweetener | 4.6% |
| Glidant | 0.3% |
| Lubricant | 1.5% |
| Wicking Agent | 5.8% |
| Flavor | 3.8% |

### Example 6

PAXIL, (−)-trans-4R-(4′-flurophenyl)-3S[(3′,4′-methylenedioxyphenoxy)methyl] piperidine hydrochloride hemihydrate, is available from GlaxoSmithKline.

This example describes the preparation of a number of fast disintegrating PAXIL formulations. In one example, the Applicant contemplates an oral dosage forms formulated as a tablet. The mass of this tablet should be less than about 1.00 g and more preferably less than about 0.80 g. In a preferred embodiment the mass of the tablet will be 250 mg. Furthermore, the gram weight of paroxetine in tablet of a given size may be varied. While it is not intended the present invention be limited specific formulation, in selected example the paroxetine in said 250 mg tablet may be adjusted to yield fast disintegrating 250 mg tablets having 10 mg, 25 mg, or 40 mg of paroxetine in each tablet. The tablet may include surface markings, cuttings, grooves, letters and or numerals for the purpose of decoration, identification, and/or designation of the number of mg of paroxetine contained therein.

Preferably, the tablet includes micro particles containing one or more systemically distributable pharmaceutical ingredients, together with an effervescent disintegrating agent. The size of the tablet will be dependent upon the amount of material used.

The term “systemically distributable pharmaceutical ingredient”, as used in examples 7 and 8, is a pharmaceutical ingredient which is conducted from the mouth to the digestive system for absorption through the stomach or intestines and systemic distribution through the bloodstream. The term is not limited to pharmaceutical ingredients which are systemically active or which systemically distribute over time. For the purposes of the instant example, a systemically distributable formulation of PAXIL. The amount of PAXIL incorporated in each tablet may be selected according to known principles of pharmacy.

The amount of effervescent disintegration agent contemplated by this example ranges from about 5 to about 50% by weight of the final composition (and preferably between about 15 and about 30% by weight thereof). In a more preferred embodiment, the amount of effervescent disintegration agent contemplated by this example ranges from between about 20 and about 25% by weight of the total composition.

More specifically, tablets contemplated by the instant example should contain an amount of effervescent disintegration agent effective to aid in the rapid and complete disintegration of the tablet when orally administered. By “rapid”, it is understood that the tablets are expected to disintegrate in the mouth of a patient in less than 10 minutes, and more desirably, between about 10 seconds and about 3 minutes.

The dosage form according to this example may further include one or more additional adjuvants which can be chosen from those known in the art including flavors, dilutents, colors, binders, filler, compaction vehicles, and non-effervescent disintegrants.

Tablets according to this example can be manufactured by known tabletting procedures. In common tabletting processes, the material which is to be tableted is deposited into a cavity, and one or more punch members are then advanced into the cavity and brought into intimate contact with the material to be pressed, whereupon compressive force is applied. The material is thus forced into conformity with the shape of the punches and the cavity. Hundreds, and even thousands, of tablets per minute can be produced in this fashion.

Materials to be incorporated in the tablets, other than the micro particles and the effervescent disintegration agent, may be pretreated to form granules that readily lend themselves to tabletting. This process is known as granulation. As commonly defined, “granulation” is any process of size enlargement whereby small particles are gathered together into larger, permanent aggregates to yield a free-flowing composition having a consistency suitable for tabletting. Such granulated compositions may have consistency similar to that of dry sand. Granulation may be accomplished by agitation in mixing equipment or by compaction, extrusion or globulation.

Lubricant, as used in examples 6 and 7, means a material which can reduce the friction arising at the interface of the tablet and the die wall during compression and ejection thereof. Lubricants may also serve to prevent sticking to the punch and, to a lesser extent, the die wall as well. The term “antiadherents” is sometimes used to refer specifically to substances which function during ejection. As used in the present disclosure, however, the term “lubricant” is used generically and includes “antiadherents”. Tablet sticking during formation and/or ejection may pose serious production problems such as reduced efficiency, irregularly formed tablets, and non-uniform distribution of intended agents or ingredients to be delivered thereby. These problems are particularly severe with high speed tabletting approaches and methods.

The PAXIL formulated according to the instant example are processed into micro particles. Each microparticle incorporates the PAXIL in conjunction with a protective material. The microparticle may be provided as a microcapsule or as a matrix-type microparticle. Microcapsules typically incorporate a discrete mass of the pharmaceutical ingredient surrounded by a discrete, separately observable coating of the protective material. Conversely, in a matrix- type particle, the pharmaceutical ingredient is dissolved, suspended or otherwise dispersed throughout the protective material. The micro particles desirably are between about 75 and 600 microns mean outside diameter, and more preferably between about 150 and about 500 microns. Micro particles above about 200 microns may be used. Thus, it is contemplated the micro particles will be between about 200 mesh and about 30 mesh U.S. standard size, and more preferably between about 100 mesh and about 35 mesh.

Although such prompt release is preferred, the protective material utilized in the microparticle, preferably, should not dissolve instantaneously in water or saliva. That is to say, the microparticle should resist dissolution and release for a period of time sufficient to permit the patient to swallow the released microcapsules as the tablet disintegrates. Micro particles made using any of the polymeric protective materials discussed below will not dissolve instantaneously.

Methods of microencapsulation are described in the Lieberman text: Pharmaceutical Dosage Form: Tablets Volume 1, Second Edition, New York, 1989, at pages 372-376 (hereby, incorporated by reference). One method taught in Lieberman is the technique of phase separation or coacervation which involves processing three mutually immiscible phases, one containing the pharmaceutical ingredient (in this example, PAXIL), another containing the protective coating material and a third containing a liquid vehicle used only in the manufacturing phase. The three phases are mixed and the protective material phase deposits by absorption on the pharmaceutical ingredient phase. After this step, the protective material phase is converted to a substantially solid form by cross-linking or by removal of solvent from this phase.

Other common techniques may be used for forming matrix-type micro particles wherein the pharmaceutical ingredient is dispersed in the protective material. For example, the pharmaceutical ingredient and a solution of a polymeric protective material may be sprayed to form droplets and contacted with a gas such as hot air so as to remove the solvent from the droplets. Such a mixture may also be dried to a solid and then comminuted to form the micro particles. Alternatively, the mixture of the pharmaceutical ingredient and polymeric solution may be mixed with an immiscible liquid phase and the solvent may be removed through this phase. The mixing step may include emulsification of the phase bearing the pharmaceutical ingredient and the protective material in the immiscible liquid phase.

The protective material may incorporate polymers such as those conventionally utilized in protective materials for micro particles. A wide variety of polymers are known for this purpose. Any such known polymeric material, utilized heretofore in production of microcapsules and/or matrix-type micro particles may be employed as a protective material in micro particles according to the instant example. Among these are cellulosic materials such as naturally occurring cellulose and synthetic cellulose derivatives; acrylic polymers and vinyl polymers. Other suitable polymers include proteinaceous materials such as gelatin, polypeptides and natural and synthetic shellacs and waxes. Particularly preferred protective material polymers include ethylcellulose, methylcellulose, carboxymethylcellulose and the acrylic resin materials sold under the registered trademark EUDRAGIT by Rhone Pharma GmbH of Weiterstadt, Germany.

Many of the protective material polymers discussed above have substantial resistance to dissolution in water. Such water-insoluble materials can be used to make delayed-release micro particles. Preferably, however, where the protective material incorporates water-insoluble materials of this nature, it also includes other ingredients to promote more rapid release of the pharmaceutical ingredient. Such release promoters include soluble polymers and, in particular, polyfractional alcohols such as mannitol, as well as magnesium oxide. For example, the acrylic material of the type known as EUDRAGIT RL30-D, when used with conventional coingredients such as methylcellulose and magnesium sterate tends to provide a slow release, typically about 50 percent or less after 30 minutes. However, a protective material incorporating the same polymeric material in conjunction with about 2 to about 4, and preferably about 2.7 parts mannitol per part EUDRAGIT material on a solids basis, and also incorporating about 0.05 to about 0.2, and preferably about 0.09 parts magnesium oxide per part EUDRAGIT solids provides a protective material with substantially immediate release properties.

Table 7 sets out ingredients suitable for the manufacture of a PAXIL containing microparticle:

TABLE 7

| Ingredient: | Grams Solids/% By Weight |
|---|---|
| EUDRAGIT RL-30-D | 210/31.7 |
| PAXIL | 17.5/2.7 |
| Mannitol | 420.0/63.5 |
| Magnesium Oxide | 14.0/2.1 |

The EUDRAGIT material will be furnished by the manufacturer as a dispersion containing 30% solids (polymer) in water. The quantity needed to provide 157.5 grams solids will be placed in a beaker and mixed to form a vortex. The mannitol and PAXIL are added and mixing is continued for 10 minutes. After this 10 minute mixing period, the magnesium oxide is added and mixing is continued for another 10 minutes. These mixing steps will take place at room temperature. The resulting mixture will be poured into a tray and dried in an oven at 50° C. under air for one hour. After one hour, the resulting partially dried mixture will be broken into lumps and then dried for an additional hour at 50° C. The dried lumps are then comminuted to micro particles, and screened through an 8 mesh screen. The screened micro particles will be dried for an additional hour at 60° C.

The fraction of the resulting micro particles passing through a 30 mesh screen will be collected. These micro particles will be tableted into an effervescent tablet of about 1.0-2.0 kilo pounds hardness with an effervescent disintegration agent and other ingredients according to the following formulation set out in Table 8:

TABLE 8

| Ingredient: | mg/Tablet |
|---|---|
| Mannitol | 225.0 mg |
| Aspartame | 40.0 mg |
| Cherry Flavor | 6.0 mg |
| Magnesium Stearate | 5.0 mg |
| Silicon Dioxide | 1.0 mg |
| Sodium Bicarbonate | 100.0 mg |
| Citric Acid | 80.0 mg |
| Micro particles | 94.3 mg |

The effervescent tablet will have a dissolution time of less than about one minute.

When administered by mouth, it will provide substantially the same bioavailability offered by conventional formulation of PAXIL. The forgoing example illustrates one embodiment of the present invention. It is not intended that the present invention be limited to any specific example. For example, the amount of PAXIL formulated in a given example may be increased or without departing from the spirit and scope of the invention.

### Example 7

This example presents another fast disintegrating formulation, as set out in Table 9, of PAXIL. The other constituents of this tablet may be selected from the ingredients described in Example 6 above.

TABLE 9
Ingredients As A Percentage Of Tablet Mass

| | |
|---|---|
| PAXIL | 5.8% |
| Powdered Mannitol | 76.2% |
| Citric Acid | 1.7% |
| Sweetener | 4.6% |
| Glidant | 0.3% |
| Lubricant | 1.5% |
| Wicking Agent | 5.8% |
| Flavor | 3.8% |
| Color | 0.3% |

(Calculated in view of 650 mg total tablet weight)

Tablets will be produced using a direct compression method as follows. All of the material, except the lubricant, will be weighed and blended for a period of between about 30 and about 50 minutes. Thereafter, the lubricant will be added and the mixture will be blended for an additional 5 to 15 minutes. The blend will then be tableted on a conventional 6 or 16 stage rotating tablet press at 25-30 revolutions per minute. Tablets are compressed using an average compression force of approximately 10.27 kN. These tablets are expected to disintegrate in between 20 and 30 seconds, without the use of an effervescent disintegrant.

### Example 8

This example presents data, in Table 10, of changes in body weight in patients taking FAZACLO between their first dose of FAZACLO and after three weeks on FAZACLO. All patients had been previously been treated with equivalent amounts of CLOZARIL or other non-rapidly disintegrating forms of clozapine. None these patients presented with any clinical findings that could otherwise explain a loss in weight. That is to say, none of these patients were prescribed additional medications, started any dieting regimes designed to decrease caloric intake or reported the onset of any new illness in the interval between the first and second “weigh in”.

TABLE 10

| | | | | Baseline | Visit 2 | Weight | | |
|---|---|---|---|---|---|---|---|---|
| Pt. # | Age | Race | Sex | Weight | Weight | Loss | Diagnosis | Dose* |
| 1 | 59 | W | M | 162 | 155 | 7 | schizoaffective | 300 |
| 2 | 58 | AA | M | 180 | 173 | 12 | schizoaffective | 400 |
| 3 | 44 | AA | M | 158 | 151 | 7 | schizoaffective | 200 |
| 4 | 48 | AA | M | 210 | 198 | 12 | schizoaffective | 300 |
| 5 | 73 | W | F | 132 | 120 | 12 | schizoaffective | 25 |
| 6 | 65 | AA | F | 172 | 165 | 7 | schizoaffective | 100 |
| 7 | 75 | AA | M | 130 | 123 | 7 | schizoaffective | 50 |
| 8 | 49 | W | M | 190 | 190 | 0 | schizoaffective | 200 |
| 9 | 70 | W | M | 170 | 163 | 7 | schizoaffective | 100 |
| 10 | 46 | W | M | 255 | 240 | 15 | schizoaffective | 300 |
| 11 | 71 | AA | F | 169 | 162 | 7 | schizoaffective | 100 |
| 12 | 54 | AA | M | 245 | 238 | 7 | schizoaffective | 300 |
| 13 | 49 | W | M | 191 | 184 | 7 | schizoaffective | 300 |
| 14 | 38 | AA | M | 184 | 172 | 12 | schizoaffective | 100 |
| Mean | 57 | | | 182 | 173.86 | 8.5 | | |
| S.D. | 12 | | | 35.98 | 35.24 | 3.74 | | |

*dose of FAZACLO in mg/day (range = 25-400 mg/day)

Other aspects, objects and advantages of this invention can be obtained from a study of the disclosure, the drawing and the appended claims.

I claim:

1. A method for reducing body weight comprising:
a. providing a patient treated with a conventional formulation of an antidepressant and;
b. treating said patient with a fast disintegrating antidepressant formulation conditions such that body weight is reduced.

2. The method, as claimed in claim 1, wherein said fast disintegrating antidepressant is selected from the group consisting of: citalopram, escitalopram oxalate, fluvoxamine maleate, paroxetine, fluoxetine, sertraline, amitriptyline, clomipramine, desipramine, doxepin, imipramine, nortryptyline, protriptyline, trimipramine, maprotiline, bupropion, buspirone, duloxetine, mirtazapine, nefazodone, reboxetine, trazodone, venlafaxine, phenelzine, tranylcypromine, and lithium carbonate.

3. A method for reducing body weight comprising:
a. providing a patient who has been taking a conventional formulation of an antidepressant for a period of greater than three weeks;
b. discontinuing administration of said conventional formulation of an antidepressant and;
c. treating said patient, after said discontinuation of said conventional formulation of an antidepressant, with a fast disintegrating formulation of a antidepressant under conditions such that body weight is reduced.

4. The method, as claimed in claim 3, wherein the interval between the discontinuation of said conventional formulation of an antidepressant and the treatment of said patient with said fast disintegrating formulation of a antidepressant is in the range between one and three days.

5. The method, as claimed in claim 3, wherein said fast disintegrating formulation of an antidepressant is selected from the group consisting of: citalopram, escitalopram oxalate, fluvoxamine maleate, paroxetine, fluoxetine, sertraline, amitriptyline, clomipramine, desipramine, doxepin, imipramine, nortryptyline, protriptyline, trimipramine, maprotiline, bupropion, buspirone, duloxetine, mirtazapine, nefazodone, reboxetine, trazodone, venlafaxine, phenelzine, tranylcypromine, and lithium carbonate.

6. A method for reducing body weight comprising:
a. providing a patient treated with a conventional formulation of a dibenzodiazapine and;
b. treating said patient with a fast disintegrating formulation of 8-Chloro-11-(1-piperazinyl)-5H-dibenzo[b,E][1,4]diazepine under conditions such that body weight is reduced.

7. A method for reducing body weight comprising:
a. providing a patient who has been taking a conventional formulation of an atypical antipsychotic for a period of greater than three weeks;
b. discontinuing administration of said conventional formulation of an atypical antipsychotic and;
c. treating said patient, after said discontinuation of said conventional formulation of an atypical antipsychotic, with a fast disintegrating formulation of 8-Chloro-11-(1-piperazinyl)-5H-dibenzo[b,E] [1,4]diazepine under conditions such that body weight is reduced.

8. The method, as claimed in claim 7, wherein the interval between the discontinuation of said conventional formulation of an atypical antipsychotic and the treatment of said patient with said fast disintegrating formulation of a 8-Chloro-1-(1-piperazinyl)-5H-dibenzo[b,E][1,4]diazepine is in the range between one and three days.

9. A method for modulating body weight comprising:
a. providing: i) a patient exhibiting at least one symptom of psychosis having a BMI of 24 or less, and ii) a fast disintegrating formulation of a clozapine metabolite and;
b. administering said fast disintegrating clozapine metabolite formulation for a period of at least four weeks under conditions such that after said fast disintegrating clozapine metabolite treatment, at least four weeks in duration, the BMI for said patient is less than 25.

10. The method, as claimed in claim 9, wherein said fast disintegrating clozapine metabolite is desmethyl clozapine.

11. A method for modulating body weight comprising:
a. providing: i) a patient exhibiting at least one symptom of depression having a BMI of 24 or less, and ii) a fast disintegrating formulation of an antidepressant and;
c. administering said fast disintegrating antidepressant formulation for a period of at least four weeks under conditions such that after said fast disintegrating antidepressant treatment, at least four weeks in duration, the BMI for said patient is less than 25.

12. The method, as claimed in claim 11, wherein said fast disintegrating antidepressant is paroxetine.
